(19) United States
(12) Patent Application Publication
(10) Pub. No.: US 2025/0283048 A1
(43) Pub. Date: Sep. 11, 2025

(54) PLATELET-ACTIVATING FACTOR BLOCKADE INHIBITS TUMOR GROWTH

(71) Applicant: The Johns Hopkins University, Baltimore, MD (US)

(72) Inventors: Stephen J. Meltzer, Baltimore, MD (US); Yulan Cheng, Baltimore, MD (US); Hua Zhao, Baltimore, MD (US)

(21) Appl. No.: 18/861,246

(22) PCT Filed: May 3, 2023
(86) PCT No.: PCT/US2023/066546

§ 371 Date: Oct. 29, 2024

Related U.S. Application Data
(60) Provisional application No. 63/338,530, filed on May 5, 2022.

(51) Int. Cl. C12N 5/071; A61K 31/551; A61P 35/00

(52) U.S. Cl. CPC C12N 5/0679; A61K 31/551; A61P 35/00; C12N 2500/38; C12N 2501/02; C12N 2501/11; C12N 2501/119; C12N 2501/155; C12N 2501/345; C12N 2501/727; C12N 2503/04; C12N 2510/00; C12N 2513/00

(57) ABSTRACT

A gastroesophageal junction (GEJ) organoid, methods of generating the same, and use of the GEJ organoid in an in vitro method of evaluating a potential anti-cancer agent are disclosed. Also disclosed are methods for treating a cancer comprising providing to a subject a Platelet Activating Factor Receptor (PTAFR) antagonist, including the PTAFR antagonist comprises WEB2086. The cancer treated can be gastroesophageal junction adenocarcinoma.

### STATEMENT REGARDING RELATED APPLICATIONS

This application claims priority to U.S. Provisional Patent Application No. 63/338,530, filed May 5, 2022, the entire contents of which are incorporated herein by reference for all purposes.

### STATEMENT OF GOVERNMENTAL INTEREST

This invention was made with government support under grant DK118250 awarded by the National Institutes of Health. The government has certain rights in the invention.

### FIELD

Provided herein are organoid models of the gastroesophageal junction, methods of making the same, and methods of using the same for in vitro evaluation of potential therapeutic agents, including anti-cancer agents.

### BACKGROUND

Gastroesophageal cancers account for more than 1 million deaths annually, representing the second-highest cause of cancer death worldwide. One category of these neoplasms, gastroesophageal junction (GEJ) adenocarcinoma, has increased its incidence over 2.5-fold in the United States and other western countries in recent decades. Compared with other gastroesophageal cancers, GEJ tumors are particularly aggressive and have dire prognosis, and therefore vigorous investigation of its molecular basis is crucially needed. This goal, however, has been notoriously difficult to achieve, in part due to a lack of biologically relevant GEJ-specific disease models.

### SUMMARY

In some aspects, the presently disclosed subject matter provides biologically relevant GEJ-specific disease models and methods of generating the same, and their use in identifying therapeutic agents for the effective treatment for cancer, including GEJ adenocarcinoma.

More particularly, in some aspects, the presently disclosed subject matter provides a gastroesophageal junction (GEJ) organoid, the organoid comprising cells wherein expression of tumor protein P53 (TP53) and cyclin dependent kinase inhibitor 2A (CDKN2A) is reduced compared to expression in a wildtype GEJ cell. In certain aspects, the organoid is capable of in vitro propagation for at least 12 months. In certain aspects, the organoid is capable of in vitro propagation for at least 18 months. In particular aspects, the organoid is generated by a method comprising culturing cells derived from the gastroesophageal junction of a subject in a medium comprising prostaglandin E 2 (PGE2), human fibroblast growth factor-10 (rFGF-10), human epidermal growth factor (hEGF), Noggin, and Gastrin I. In more particular aspects, the medium further comprises one or more inhibitors, wherein the one or more inhibitors are selected from ALK5 inhibitor A-83-01, p38 MAPK inhibitor SB202190, Rho-associated, coiled-coil containing protein kinase (ROCK) inhibitor Y27632, and GSK3 inhibitor CHIR99021. In more particular aspects, the medium further comprises one or more supplements selected from antibiotics, N-acetylcysteine, and Nicotinamide. In certain aspects, expression of TP53 and CDKN2A is reduced by CRISPR-mediated gene inactivation or siRNA mediated gene silencing.

In other aspects, the presently disclosed subject matter provides a method of generating a gastroesophageal junction organoid, the method comprising culturing cells obtained from the gastroesophageal junction of a subject in a suitable medium to promote cell growth and/or differentiation; and reducing expression of tumor protein P53 (TP53) and cyclin dependent kinase inhibitor 2A (CDKN2A) in the cells.

In certain aspects, the subject is a human. In particular aspects, expression of TP53 and CDKN2A is reduced by CRISPR-mediated gene inactivation. In more particular aspects, expression of TP53 and CDKN2A is reduced by transfecting cells with a TP53 crRNA comprising the sequence CCCCGGACGATATTGAACAA (SEQ ID NO: 1), a CDKN2A crRNA comprising the sequence CCCAACGCACCGAATAGTTA (SEQ ID NO: 2), and a nuclease. In yet more particular aspects, the nuclease comprises Cas9 endonuclease. In certain aspects, expression of TP53 and CDKN2A is reduced by si-RNA mediated gene silencing. In more certain aspects, the medium comprises prostaglandin E 2 (PGE2), human fibroblast growth factor-10 (rFGF-10), human epidermal growth factor (hEGF), Noggin, and Gastrin I.

In certain aspects, the medium further comprises one or more inhibitors, wherein the one or more inhibitors are selected from ALK5 inhibitor A-83-01, p38 MAPK inhibitor SB202190, Rho-associated, coiled-coil containing protein kinase (ROCK) inhibitor Y27632, and GSK3 inhibitor CHIR99021. In more certain aspects, the medium further comprises one or more supplements selected from antibiotics, N-acetylcysteine, and Nicotinamide.

In other aspects, the presently disclosed subject matter provide a GEJ organoid for use in an in vitro method of evaluating a potential anti-cancer agent.

In yet other aspects, the presently disclosed subject matter provides an in vitro method of evaluating a potential anti-cancer agent, the method comprising contacting the presently disclosed GEJ organoid with the potential anti-cancer agent; and measuring a response in the organoid.

In certain aspects, measuring a response in the organoid comprises measuring organoid size, cell viability, and/or one or more markers of cell proliferation. In particular aspects, a decreased organoid size, a decreased cell viability, and/or decreased expression of one or more markers of cell proliferation following contacting the organoid with the potential anti-cancer agent indicates a positive response to the agent.

In other aspects, the presently disclosed subject matter provides a method of treating cancer in a subject, comprising providing to the subject a Platelet Activating Factor Receptor (PTAFR) antagonist. In particular aspects, the PTAFR antagonist comprises WEB2086. In certain aspects, the cancer is gastroesophageal junction adenocarcinoma.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one figure executed in color. Copies of this patent or patent application publication with color figures will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D show the establishment and characterization of human normal GEJ organoids. (FIG. 1A) A workflow of organoid generation from human primary endoscopic GEJ biopsies. Biopsies of normal GEJ mucosa were taken by upper endoscopy, and then minced and enzymatically dissociated. The cell suspension was mixed with Matrigel to initiate 3D organoid culture in conditioned medium. (FIG. 1B-FIG. 1D) GEJ organoids were analyzed for structural and growth properties at the indicated time points. 3D organoids were photomicrographed under phase-contrast microscopy (FIG. 1B, upper panel) and collected for H&E staining (FIG. 1B, lower panel). Scale bar=50 μm. Average organoid size (FIG. 1C) and (FIG. 1D) viability were determined at each 6 timepoint. *, P<0.05 vs. Day 1; #, P<0.05 vs. Day 14; ns, not significant vs. Day 24. Data represent 6 biological replicates;

FIG. 2A, FIG. 2B, FIG. 2C, FIG. 2D, FIG. 2E, FIG. 2F, and FIG. 2G show that knockout of TP53/CDKN2A promotes neoplastic transformation in human normal GEJ organoids. Cas9 nuclease and the negative control or TP53/CDKN2A-targeted gRNA complex were transfected into GEJ organoids (FIG. 2A and FIG. 2B) DNA sequencing of TP53/CDKN2A^{KO} GEJ organoids showing 1-bp insertion or deletion. Red font indicates corresponding frameshift indels in the DNA. (FIG. 2C and FIG. 2D) On the 10th day after seeding 1×10⁵ dissociated organoid cells, organoid cultures were photomicrographed using phase-contrast microscopy and collected for (FIG. 2C) H&E and (FIG. 2D) IF staining for Ki-67 (red color). (FIG. 2E) Average organoid size, organoid-forming efficiency, and Ki-67 index were determined by measuring >50 organoids. *P<0.05. (FIG. 2F) Representative images of xenografts from mice injected with control or TP53/CDKN2A^{KO} GEJ organoids; the underlying table shows quantification and tumor characteristics. (FIG. 2G) Representative H&E and AE1/AE3 pan-keratin IHC staining (brown) in xenografts arising from TP53/CDKN2A^{KO} organoids. White arrows, mitoses; white circle, abnormally large nucleus; scale bar=100 μm;

FIG. 3A, FIG. 3B, FIG. 3C, and FIG. 3D show that platelet-activating factors (PTAFs) are among the most upregulated lipids in TP53/CDKN2A^{KO} vs. control GEJ organoids. (FIG. 3A) Matrix-assisted laser desorption/ionization (MALDI) imaging-based lipidomic analysis was performed on independent paired sets of TP53/CDKN2A^{KO} vs. control GEJ organoids derived from two different patients. (FIG. 3B) The most discriminative peaks (m/z signals) according to fold-change are shown. Venn diagram represents the overlap of upregulated and downregulated lipids based on AUC and Volcano plot results. Overlapping 16 upregulated lipids and AUC values are listed. (FIG. 3C and FIG. 3D) MALDI imaging and corresponding chemical structures of representative PTAFs.

FIG. 4A, FIG. 4B, FIG. 4C, FIG. 4D, FIG. 4E, FIG. 4F, FIG. 4G, and FIG. 4H shows that blockade of PTAF/PTAFR inhibits growth and proliferation, and in vivo tumorigenesis of TP53/CDKN2A^{KO} GEJ organoids. (FIG. 4A) mRNA levels of PTAFR in TP53/CDKN2A^{KO} vs. control GEJ organoids (left) and normal GEJ vs. EAC tissues. (FIG. 4B) Knockdown of PTAFR mRNA by siRNAs in TP53/CDKN2A^{KO} GEJ organoids. (FIG. 4C) Average organoid size and (E) cell viability determined by phase-contrast imaging and WST1 assays, respectively. (FIG. 4D) Ki67 labeling images and (FIG. 4E) quantification obtained on Day 7. *P<0.05 vs. same-day siCtrl. Scale bar=100 μm. (FIG. 4F-FIG. 4H) Average organoid size and cell viability (H) determined by phase-contrast imaging and WST1 assays, respectively. (FIG. 4G and FIG. 4H) Ki67 labeling images and quantification obtained on Day 10. Scale bar=100 μm. *P<0.05 vs Ctrl-DMSO on the same day; +, P<0.05 vs. WEB 10 μM on the same day; #P<0.05 vs. WEB 15 μM on the same day;

FIG. 5A, FIG. 5B, FIG. 5C, FIG. 5D, and FIG. 5E shows that blockade of PTAF/PTAFR inhibits in vivo tumorigenesis of TP53/CDKN2A^{KO} GEJ organoid and EAC cell line Eso26. (FIG. 5A) Xenograft images and incidences after WEB2086 treatment in TP53/CDKN2A^{KO} GEJ organoids. Scale bar=1 cm. (FIG. 5B) Representative H&E and AE1/AE3 pan-keratin IHC staining (brown) in xenografts arising from TP53/CDKN2A^{KO} organoids. White arrows, mitoses; white circles, abnormally large nucleus; scale bar=100 μm. (FIG. 5C) Xenograft images and incidences after WEB2086 treatment in Eso26 cell line. Scale bar=1 cm. (FIG. 5D) si *P<0.05. (FIG. 5E) H&E and IHC staining analysis of Ki67 in Eso26 xenograft samples. Scale bar=100 μm;

FIG. 6A, FIG. 6B, FIG. 6C, FIG. 6D, FIG. 6E, and FIG. 6F show that FOXM1 is a prominently enriched important transcription factor in TP53/CDKN2A^{KO} GEJ organoids. (FIG. 6A) Analyses of comprehensive RNA-seq data showed the distribution of differentially expressed genes (DEGs) in TP53/CDKN2A^{KO} organoids vs. control organoids. A cut-off adjusted p (padj) value of 2 were chosen to define significant DEGs. (FIG. 6B) Gene ontology (GO) analysis identified the top-ranking key terms as biological processes and cellular components as being significantly associated with transcriptional profiles in TP53/CDKN2A^{KO} organoids. (FIG. 6C and FIG. 6D) Global DNA methylation analysis in TP53/CDKN2A^{KO} and control GEJ organoids derived from patient 1. Volcano plots (FIG. 6C) revealed differentially methylated regions (DMRs), defined as exceeding a cutoff p-value of <0.05 and an absolute 20 delta-methylation value of >0.2. Hypormethylated regions (FIG. 6D) were confirmed in TP53/CDKN2A^{KO} or control organoids, respectively. (FIG. 6E) Top-ranked transcription factors belonging to Forkhead box (FOX) family binding motifs enriched in TP53/CDKN2A^{KO} vs. control GEJ organoids. P-values were calculated by the HOMER package. (FIG. 6F) Expression analysis of FOX family TFs in TP53/CDKN2A^{KO} vs. control organoids and in EAC vs. normal GEJ tissues from the TCGA database. Genes are ranked based on the −log 10 (p-value) of EAC tissue vs. normal GEJ expression levels. Size indicates −log 10 (p-value); color indicates direction of enrichment in the comparison; missing bubbles indicate not detected; and

FIG. 7A, FIG. 7B, FIG. 7C, FIG. 7D, FIG. 7E, FIG. 7F, FIG. 7G, and FIG. 7H show that PTAFR is a direct downstream target of FOXM1. (FIG. A) Motif enrichment analysis of PTAFR promoter region. (FIG. 7B) ChIP-seq profiles for H3K4me and FOXM1 at the PTAFR locus in indicated tissues and cell lines. (FIG. 7C) Knockdown of FOXM1 by siRNAs reduced PTAFR mRNA transcript levels in TP53/CDKN2A^{KO} GEJ organoids. (FIG. 7D) Schematic of PTAFR transcriptional start site (TSS) (green) and 0 to 1200 bp upstream sequence. PCR primers for ChIP experiments (orange horizontal lines) and FOXM1 binding motifs (red vertical lines) are indicated. (FIG. 7E) ChIP-qPCR detection of FOXM1 occupancy at the PTAFR promoter region, using primer sets indicated in (FIG. 7D). ChIP was performed with either antiFOXM1 or anti-IgG antibodies, and fold enrichment relative to anti-IgG is shown. (FIG. 7F-FIG. 7H) Average organoid size determined by phase-contrast imaging and cell viability determined by WST-1 assays, respectively. Ki67 labeling images and quantification obtained on Day 7. Scale bar=100 μm. *, P<0.05 vs. siCtrl on the same day.

FIG. 8A, FIG. 8B, FIG. 8C, and FIG. 8D show TP53/CDKN2A^{KO} GEJ organoids created by CRISPR-Cas9 genome editing. (FIG. 8A) Electroporation preparation workflow and (FIG. 8B) two-step electroporation settings. (FIG. 8C) Cas9 nuclease and the negative control or TP53/CDKN2A-targeted gRNA complex were successfully transfected into GEJ organoids (red fluorescence). (FIG. 8D) survival and sustained growth of TP53/CDKN2A^{KO} GEJ organoids in Nutlin-3a-containing selective medium. Scale bar=100 μm.

FIG. 9A-FIG. 9B show average lipid spectra and MALDI images of control and TP53/CDKN2A^{KO} GEJ organoids. (FIG. 9A) Average lipid mass spectra collected from an organoid section. Inset shows detail and complexity of the spectra (typical tissue imaging experiments result in up to thousands of such spectra). (FIG. 9B) Ion images generated from each peak. Each m/z value of interest is displayed as relative intensity.

FIG. 10 shows 4 PTAF lipids and a PTAF lipid precursor identified by MS/MS and fragmentation analysis.

FIG. 11 shows PTAFR mRNA levels in human normal GEJ vs. EAC tissue samples.

FIG. 12A, FIG. 12B, and FIG. 12C show global DNA methylation analysis in TP53 CDKN2A^{KO} and control GEJ organoids derived from three different patients. Volcano plots (up) revealed differentially methylated regions (DMRs), defined as exceeding a cutoff p-value of <0.05 and an absolute delta-methylation value of >0.2. Hypomethylated regions (lower 3 panels) were confirmed in TP53 CDKN2A^{KO} and control organoids, respectively.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof).

As used herein, the terms “prevent,” “prevention,” and preventing” refer to reducing the likelihood of a particular condition or disease state from occurring in a subject not presently experiencing or afflicted with the condition or disease state. The terms do not necessarily indicate complete or absolute prevention. For example “preventing” refers to reducing the likelihood of a condition or disease state occurring in a subject not presently experiencing the condition or disease.

The terms “subject” and “patient” are used interchangeably herein and refer to any animal. In some embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human In some aspects, the human is an adult aged 18 years of older. In some aspects, the human is a child aged 17 years or less.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Described herein are human gastroesophageal junction (GEJ)-derived organoids, modified by TP53/CDKN2A dual-knockout (TP53/CDKN2A^{KO}) achieved via CRISPR/Cas9 genome editing. Compared with their wild-type counterparts, TP53/CDKN2A^{KO} organoids are more proliferative, larger in size, more dysplastic in morphology, more likely to form tumors in vivo, and distinguished by a markedly altered lipidomic profile. The most upregulated lipids, Platelet-Activating Factors (PTAFs), exert strong pro-growth functions in GEJ organoids by activating its cognate receptor, PTAFR. TP53/CDKN2A dual inactivation also causes disruption of both the transcriptome and the methylome, likely mediated by key transcription 4 factors, particularly FOXM1. FOXM1 activates PTAFR transcription by binding to the PTAFR promoter, further amplifying the PTAF-PTAFR pathway. Taken together, these findings establish a robust model system for investigating early GEJ neoplastic events, identify crucial metabolic and epigenomic changes during GEJ tumorigenesis, and provide novel insights into pro-neoplastic mechanisms associated with TP53/CDKN2A inactivation in GEJ neoplasia.

In some embodiments, provided herein are systems and methods for modeling a disease state. The systems described herein find use in modeling a disease state to understand molecular mechanisms underlying the disease state. The systems described herein further find use in determining efficacy of a potential therapeutic agent for the disease state.

In some embodiments, the systems provided herein are organoids. The term “organoid” as used herein refers to an artificially produced mass of cells or tissues that resembles an organ. In some embodiments, an “organoid” refers to a three-dimensional construct grown in vitro that mimics one or more properties of an organ. In some embodiments, an organoid is derived from stem cells that are differentiated into desired cell types and organize into a desired structure. Human organoids are robust models that recapitulate and maintain essential genetic, functional and phenotypic characteristics of initial tissues. Filling the gap between transgenic mice model and classic cell lines, organoids offer a valuable opportunity to investigate the fundamental mechanism of oncogenic events as well as improve therapy.

In some embodiments, the systems described herein are disease-specific organoids. The term “disease-specific” refers to an organoid that models a given disease state or condition in a subject. For example, a disease-specific organoid may be derived from cells obtained from a subject afflicted with or suffering from the desired disease. For example, a cancer-specific organoid may be developed starting from cells obtained from a subject afflicted with cancer.

In some embodiments, provided herein is a disease-specific organoid that models gastroesophageal junction (GEJ) adenocarcinoma. In some embodiments, the organoid is derived from a biopsy of the GEJ of a subject. The term “gastroesophageal junction” or “GEJ” as used interchangeably herein refers to an anatomical area where the esophagus (e.g., the distal esophagus) joins the stomach (e.g., the proximal stomach, also referred to as the cardia). Accordingly, a biopsy of the GEJ may contain tissue from the GEJ, along with tissue from the neighboring regions (e.g. the distal esophagus and the proximal stomach). In some embodiments, the organoid is derived from a subject suffering from GEJ adenocarcinoma. In some embodiments, the organoid is derived from a biopsy of a control subject, and expression of one or more genes in the organoid is modulated in order to mimic a given disease state, such as GEJ adenocarcinoma.

In some embodiments, provided herein is a gastroesophageal junction organoid. In some embodiments, the organoid comprises cells (e.g. cells obtained from the gastroesophageal junction of a subject) wherein expression of tumor protein P53 (TP53) and cyclin dependent kinase inhibitor 2A (CDKN2A) is reduced. For example, expression of TP53 and CDKN2A may be reduced compared to expression in a wildtype GEJ cell or a wild-type GEJ organoid. The term “wildtype” when used in reference to an organoid indicates that the gene expression within the organoid has not been modulated (e.g. has not been modulated by siRNA gene silencing, CRISPR-mediated gene editing, and the like). A “wildtype” GEJ organoid, however, may be derived from a control subject or from a subject afflicted with a given disease state, such as GEJ adenocarcinoma.

The GEJ organoids described herein may be generated culturing cells obtained from the gastroesophageal junction of a subject, in a suitable medium to promote cell growth and/or differentiation, and reducing expression of TP53 and CDKN2A in the cells.

In some embodiments, the cells are obtained from a human subject. In some embodiments, the subject is a healthy subject (e.g., a subject not afflicted with a condition or disease state), also referred to herein as a “control” subject. In some embodiments, the subject is afflicted with a condition or a disease, such as cancer. In some embodiments, the cells are obtained from the gastroesophageal function of a subject afflicted with or at risk of developing GEJ adenocarcinoma.

In some embodiments, the cells are cultured in a suitable medium to promote cell growth and/or differentiation. In some embodiments, the medium comprises one or more proteins. For example, in some embodiments the medium comprises one or more growth factors. In some embodiments, the medium comprises one or more of prostaglandin E 2 (PGE2), human fibroblast growth factor-10 (rFGF-10), human epidermal growth factor (hEGF), Noggin, and Gastrin I. In some embodiments, the medium comprises at least 2, at least 3, at least 4, or each of prostaglandin E2 (PGE2), human fibroblast growth factor-10 (rFGF-10), human epidermal growth factor (hEGF), Noggin, and Gastrin I.

In some embodiments, the medium further comprises one or more inhibitors. For example, in some embodiments the growth medium further comprises one or more kinase inhibitors. For example, in some embodiments the medium further comprises one or more of: an ALK5 inhibitor, a p38 MAPK inhibitor, a Rho-associated, coiled-coil containing protein kinase (ROCK) inhibitor, and a GSK3 inhibitor. In some embodiments, the medium comprises an ALK5 inhibitor, a p38 MAPK inhibitor, and a ROCK inhibitor. In some embodiments, the medium comprises an ALK5 inhibitor, a p38 MAPK inhibitor, a ROCK inhibitor, and a GSK3 inhibitor. In some embodiments, the medium further comprises the ALK5 inhibitor A-83-01, the p38 MAPK inhibitor SB202190, and the ROCK inhibitor Y27632. In some embodiments, the medium further comprises the ALK5 inhibitor A-83-01, the p38 MAPK inhibitor SB202190, the ROCK inhibitor Y27632, and the GSK3 inhibitor CHIR99021.

In some embodiments, the medium further comprises one or more supplements. Suitable supplements include antibiotics, vitamins, antioxidants, and the like. In some embodiments, the medium further comprises an antibiotic, N-acetylcysteine, and Nicotinamide. In some embodiments, the antibiotic is penicillin, streptomycin, and/or primocin.

The medium may be changed at any suitable frequency during culture of the organoid. For example, the medium may be changed daily, every other day, every 3 days, every 4 days, every 5 days, every 6 days, weekly, and the like. In some embodiments, the medium is changed every 3 days.

In some embodiments, the organoid comprises cells wherein expression of tumor protein P53 (TP53) and cyclin dependent kinase inhibitor 2A (CDKN2A) is reduced. A reduction in expression may indicate that modulation occurs at the gene level (e.g. mRNA level) and/or at the protein level. For example, reducing expression of TP53 or CDK2NA may refer to modulating mRNA encoding TP53 or CDK2NA. In some embodiments, “reducing expression” of TP53 or CDK2NA refers to circumstances where one or more mutations are inserted into the gene sequence (e.g. mRNA sequence) for the respective gene, thereby causing a mutant protein to be produced during translation rather than the wildtype form. The TP53 gene encodes the protein tumor protein 53. Accordingly, reducing expression of TP53 may refer to reducing expression of the TP53 gene and/or protein. The CDKN2A gene provides instructions for making several proteins, including p16 (INK4A) and p14 (ARF) proteins. Accordingly, reducing expression of CDKN2A may reduce expression of the gene, and/or reduce protein levels of any protein encoded by the CDK2NA gene.

The term “reduced” as used herein in reference to expression of a gene or a protein indicates that the expression of the gene or a protein encoded by the gene is less than its expression in a control cell. Expression may be reduced by any suitable amount. Reduction does not necessarily indicate complete elimination of the gene or the protein, although the term “reduction” encompasses a complete elimination of gene expression. Accordingly, “reduced” expression of TP53 and CDKN2A may indicate that expression of these genes is “knocked out”, which may also be referred to herein as “genome editing” or “gene editing”. Alternatively, “reduced” expression may indicate that expression of the gene is “knocked down”, which may also be referred to as “gene silencing”. As described above, in some embodiments a gene may be edited/silenced by inserting one or more mutations into the gene sequence (e.g. mRNA sequence), such as by using CRISPR-based gene editing techniques, thereby reducing production of a wildtype protein encoded by the gene and promoting production of a mutant protein, which may possess diminished activity compared to the wildtype protein encoded by the un-modified gene.

Expression of TP53 and CDKN2A may be reduced in the cells using any suitable method. In some embodiments, gene expression of TP53 and CDKN2A is reduced using CRISPR-based gene editing (e.g. CRISR-based gene inactivation). CRISPR-based gene inactivation may involve the use of a guide RNA (gRNA) and an endonuclease. In some embodiments, CRISPR-based gene editing involves transfecting cells with a guide RNA and an endonuclease. Depending on the endonuclease used, in some embodiments the guide RNA comprises a CRISPR RNA (crRNA), or a crRNA and a tracrRNA. In some embodiments, the organoids described herein are generated by transfecting cells (e.g. transfecting cells obtained from the GEJ of a subject) with a TP53 crRNA comprising the sequence CCCCGGACGATATTGAACAA (SEQ ID NO: 1) and a CDKN2A crRNA comprising the sequence CCCAACGCACCGAATAGTTA (SEQ ID NO: 2). In some embodiments, the cells are additionally transfected with a tracrRNA. The use of the tracrRNA depends on which endonuclease is used. In some embodiments, the endonuclease comprises Cas9. In such embodiment, a tracrRNA is used. Other suitable Cas endonucleases may be used including Cas 3, Cas12a, Cas12d, Cas12e, Cas13, and Cas14. In some embodiments, CRISPR-based gene editing can be used to insert one or more mutations into the mRNA sequence for TP53 and/or CDKN2A gene, thereby causing translation of a mutant form of one or more proteins encoded by the gene(s) which have diminished activity compared to the wildtype protein.

In some embodiments, expression of TP53 and CDKN2A is reduced by other suitable techniques, including RNA interference, RNA editing, antisense oligonucleotides, and the like. For example, suitable RNA interference methods include the use of small interfering RNA (siRNAs) or small hairpin RNAs (shRNAs) to disrupt gene expression.

In some embodiments, the GEJ organoids described herein are capable of in vitro propagation for at least 12 months. The term “propagation” indicates that the cells remain viable, cell growth and division occurs, and/or the organoid grows in culture. In some embodiments, the GEJ organoids described herein are capable of in vitro propagation for at least 12 months, at least 13 months, at least 14 months, at least 15 months, at least 16 months, at least 17 months, at least 18 months, at least 19 months, at least 20 months, at least 21 months, at least 22 months, at least 24 months, or at least 2 years. In some embodiments, the GEJ organoids described herein are capable of in vitro propagation for at least 18 months.

The GEJ organoids described herein find use in in vitro methods for evaluating potential anti-cancer agents. For example, the organoids described herein may be used to evaluate whether an agent has anti-cancer activity, including changing molecular signaling pathways within a cell, diminishing neoplastic potential of cells, decreasing cell viability, arresting cell growth, and the like. Accordingly, in some embodiments the GEJ organoids described herein may be used in a method of evaluating a potential anti-cancer agent, comprising contacting the organoid with the potential anti-cancer agent, and measuring a response in the organoid. The response may be any one or more of organoid size, cell viability, and markers of cell proliferation. Suitable markers of cell proliferation include, for example, Ki67, proliferating cell nuclear antigen (PCNA), minichromosome maintenance (MCM) proteins, and the like. Any one or more markers of cell proliferation may be evaluated. In some embodiments, the marker of cell proliferation is Ki67. In some embodiments, Ki67 is measured as the number of Ki67 antigen positive cells, which is also described herein as the Ki67 labeling index. In some embodiments, decreased organoid size, decreased cell viability, and/or decreased expression of one or more markers of cell proliferation following contacting the organoid with the potential anti-cancer agent indicates a positive response to the agent. A positive response to the agent indicates that the agent possesses anti-cancer activity, and represents a viable candidate agent for cancer treatment in a cell or a subject.

In some aspects, provided herein is a method of treating cancer in a subject. In some embodiments, the cancer is a gastric cancer, esophageal cancer, or stomach cancer. In some embodiments, the cancer is a cancer of the gastroesophageal junction. In some embodiments, the cancer is gastroesophageal junction adenocarcinoma. The method comprises providing to the subject an antagonist of Platelet Activating Factor Receptor (PTAFR). The PTAFR antagonist may comprise any suitable agent that decreases activity and/or expression of PTAFR, including small molecules, peptides, antibodies, aptamers, and the like. In some embodiments, the PTAFR antagonist comprises WEB2086. WEB2086 is a potent PTAFR antagonist having the formula C₂₂H₂₂N₅O₂SCl. The chemical name is 4-[3-[4 [(2-Chlorophenyl)-9-methyl-6H-thieno[3,2-f][1,2,4]triazolo[4,3-α]diazepin-2-yl]-1-oxopropyl]morpholine, and the structure is shown below:

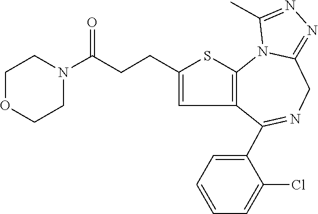 [SMILES: Cc1nnc2n1-c1sc(CCC(=O)N3CCOCC3)cc1C(c1ccccc1Cl)=NC2]

The PTAFR antagonist may be administered to the subject by any suitable route, including oral and parenteral routes (e.g., injection). The PTAFR antagonist may be administered to the subject in combination with one or more additional anti-cancer therapies, including chemotherapy, radiotherapy, immunotherapy, surgery, and the like. The PTAFR antagonist may be provided to the subject at any suitable dose, for any suitable duration, to achieve the intended effect. In some embodiments, the dose of the PTAFR antagonist (e.g., WEB2086) is about 1 μg/kg body weight to about 100 mg/kg body weight.

Other PTAFR agonists include, but are not limited to, Israpafant (Y-24180), and Rupatadine.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods

KEY RESOURCES TABLE

| | | |
|---|---|---|
| REAGENT or RESOURCE | SOURCE | IDENTIFIER |

Antibodies

| | | |
|---|---|---|
| Anti-pan Cytokeratin Antibody | Santa Cruz | Cat# sc-81714 |
| (AE 1/AE3) | | |
| Anti-KI67 antibody | Abcam | Cat# ab16667 |
| FOXM1 Recombinant Rabbit | Invitrogen | Cat# 702684 |
| Monoclonal Antibody (1H24L2) | | |
| normal mouse IgG | Merck | Cat# 12-371B |
| | Millipore | |
| Horseradish peroxidase-conjugated | GE Healthcare | Cat# NA934 |
| secondary antibody | | |
| Goat anti-Rabbit IgG Secondary | ThermoFisher | Cat# A-11036 |
| Antibody, Alexa Fluor 568 | Scientific | |

Chemicals, peptides, and recombinant proteins

| | | |
|---|---|---|
| Penicillin/Streptomycin Mixture | Quality | Cat# 120-095- |
| | Biological | 721 |
| G418 | Invitrogen | Cat# 10131035 |
| Zeocin | Invitrogen | Cat# 45-0430 |
| Collagenase from Clostridium | Sigma-Aldrich | Cat# C7657 |
| Histolyticum | | |
| Dispase #1 | ThermoFisher | Cat#1 7105041- |
| | Scientific | 5G |
| CHIR99021 | Sigma-Aldrich | Cat# SML1046 |
| PGE2 | Sigma-Aldrich | Cat# P0409 |
| Recombinant Human FGF-10 | Pepro tech | Cat# 100-26 |
| REGF | Sigma-Aldrich | Cat# E9644 |
| Recombinant Murine Noggin | Pepro tech | Cat# 250-38 |
| N-Acetyl-L-cysteine | Sigma-Aldrich | Cat# A9165 |
| Nicotinamide | Sigma-Aldrich | Cat# NO638 |
| [Gly18]-Gastrin 1-17 human | Sigma-Alirich | Cat# SCP0150 |
| A 83-01 | Sigma-Aldrich | Cat# SML0788 |
| SB 202190 | Sigma-Aldrich | Cat# 87067 |
| Y-27632 dihydrochloride | Sigma-Aldrich | Cat# Y0503 |
| Primocin | InvivoGen | Cat# ant-pm-1 |
| B-27 Supplement (50X) | ThermoFisher | Cat# 17604044 |
| | Scientific | |
| TrypLE Express Enzyme (1X) | ThermoFisher | Cat# 12604013 |
| | Scientific | |
| Bovine Serum Albumin solution | Sigma-Aldrich | Cat# A8412 |
| GlutaMAX Supplement | ThermoFisher | Cat# 35060061 |
| | Scientific | |
| HEPES | ThermoFisher | Cat# 15630080 |
| | Scientific | |
| Matrigel Matrix | Coming | Cat# 356231 |
| All-R ® S.p. Caso Nuclease V3 | IDT | Cat# 1081059 |
| BTXpress electroporation buffer | Harvard | Cat# 45-0805 |
| | Apparatus | |
| Nutlin-3a | sigma-Aldrich | Cat# SML0680 |
| Fluoroshied with DAPI | Sigma Aldrich | Cat# SLCD7376 |
| Cell Recovery Solution | Coming | Cat# 354253 |
| M-1 Embedding Matrix | ThermoFisher | Cat# 1310 |
| | Scientific | |
| WEB2086 | Sigma-Aldrich | Cat# SML0238 |

Critical commercial assays

| | | |
|---|---|---|
| Alt-R CRISPR-Cas9 Control Kit | IDT | Cat# 1072554 |
| Cell Proliferation Reagent WST-1 | Roche | Cat# 5015944001 |
| assay Kit | | |
| DNeasy Blood & Tissue Kit | Qiagen | Cat# 69506 |
| Qlagen RNeasy Mini Kit | Qiagen | Cat# 74106 |
| TOPO ™ TA Cloning ™ Kit | ThermoFisher | Cat# K450002 |
| | Scientific | |
| QIAprep Spin Miniprep Kit | Qiagen | Cat# 27104 |
| Qubit dsDNA BR Assay | ThermoFisher | Cat# Q32850 |
| | Scientific | |
| NEBNet Ultra Directional RNA | NEB | Cat# E74208 |
| Library Prep Kit | | |
| IScript Select cDNA Synthesis Kit | Bio-Rad | Cat# 1708898 |
| ITaq Universal SYBR Green Supermix | Bio-Rad | Cat# 1725120 |
| EZ-Magna ChIPTM A/G Chromatin | Merck | Cat# 17-10088 |
| Immunoprecipitation Kit | Millipore | |

Oligonucleotides

| | | |
|---|---|---|
| TP53 | IDT | N/A |
| CRNA:CCCCGGACGATATTGAACAA | | |
| CDKN2A crRNA: | IDT | N/A |
| CCCAÅCGCACCGAATAGTTA | | |
| AR-RO CRISPR-Cas9 tracrRNA, | IDT | Cat# 1075928 |
| ATTO ™ 550 | | |
| AIL-RD CRISPR-Cas9 N gative | IDT | Cat# 1072544 |
| Control crRNA #1 | | |
| PTAFR SIRNA pool | Sants Cruz | Cat# sc-40165 |
| FOXM1 SIRNA SMARTpool | Dharmacon | Cat# SO-2743818G |
| On TARGETplus Non-targeting | Dharmacon | Cat#D001810- |
| siRNA pool | | 10-05 |
| TP53 forward primer: | This study | N/A |
| GCTGCCCTGGTAGGTTTTCT | | |
| TP53 reverse primer: | This study | N/A |
| GAGACCTGTGGGAAGCGAAA | | |
| CDKN2A forward primer: | This study | N/A |
| TAACTATTCGGTGCGTTGGG | | |
| CDKN2A reverse primer: | This study | N/A |
| CTCCAGAGGATITGAGGGACAG | | |
| PTAFR forward primer: | This study | N/A |
| CCTTGTTGAGTCAGCCTCTT | | |
| PTAFR reverse primer: | This study | N/A |
| CGTGTCCATCAGTACATCA | | |
| FOXM1 forward primer: | This study | N/A |
| TCTGCCAATGGCAAGGTCTCCT | | |
| FOXM1 reverse primer: | This study | N/A |
| CTGGATTOGGICGTTTCTGCTG | | |
| HNF4A forward primer: | This study | N/A |
| CAGAATGAGCGGGACCGGATC | | |
| HNF4A reverse primer: | This study | N/A |
| CAGCAGCTGCTOCTTCATGGAC | | |
| ELF3 forward primer: | This study | N/A |
| GCTGGAAGGAGGAAGAGGTT | | |
| ELF3 reverse primer: | This study | N/A |
| CTCGAGTGGTCCGTGÅGTTT | | |
| GATA6 forward primer: | This study | N/A |
| CTCAGTTOCTACGCTTCGCAT | | |
| GATA6 reverse primer: | This study | N/A |
| GTOGAGGTCAGTGAACAGCA | | |
| KLF5 forward primer: | This study | N/A |
| ATCAAGACAGAACOTGTTGCC | | |
| KLF5 reverse primer: | This study | N/A |
| CAGCCTTCCCAGGTACACTTG | | |
| β-actin forward primer: | This study | N/A |
| ATCAAGACAGAACCTGTTGCC | | |
| β-actin reverse primer: | This study | N/A |
| CAGCCTTOCCAGGTACACTTG | | |
| PTAFR promoter set1 | This study | N/A |
| forward primer: | | |
| TAAGCCTGGGACACTCAAC | | |
| PTAFR promoter set1 | This study | N/A |
| reverse primer: | | |
| CACACATACTCCCTACCTCAC | | |

Software and algorithms

| | | |
|---|---|---|
| SCILS Lab, 2020a | SCILS GmbH | N/A |
| GenomeStudio | Illumina | N/A |
| R-3.6.0 | R Core Team, | N/A |
| | 2008 | |
| ImageJ | Open | N/A |
| GraphPad Prism 7 | GraphPad | N/A |

### Data and Code Availability

RNA-seq datasets of TCGA EAC and normal GEJ tissues can be obtained from XENA data portal (http://xena.ucsc.edu/).

### Patient Samples

In accordance with approved Institutional Review Board protocols at the Johns Hopkins Hospital, primary human endoscopic biopsy samples were acquired at the Johns Hopkins Hospital under written informed consent. Tissue samples were pathologically confirmed as nondysplastic GEJ.

### Cell Lines and Maintenance

LWnt-3A cells (CRL-2647) were purchased from ATCC and maintained in DMEM-10% FBS to produce Wnt-3A-conditioned medium. Cultrex HA-R-Spondin1-Fc 293T cells (3710-001-01) were purchased from Bio-techne and maintained in DMEM-10% FBS to generate R-spondin-1-conditioned medium. Eso26 cells were purchased from JENNIO Biological Technology and grown in RPMI 1640-10% FBS.

### GEJ Organoid Cultures

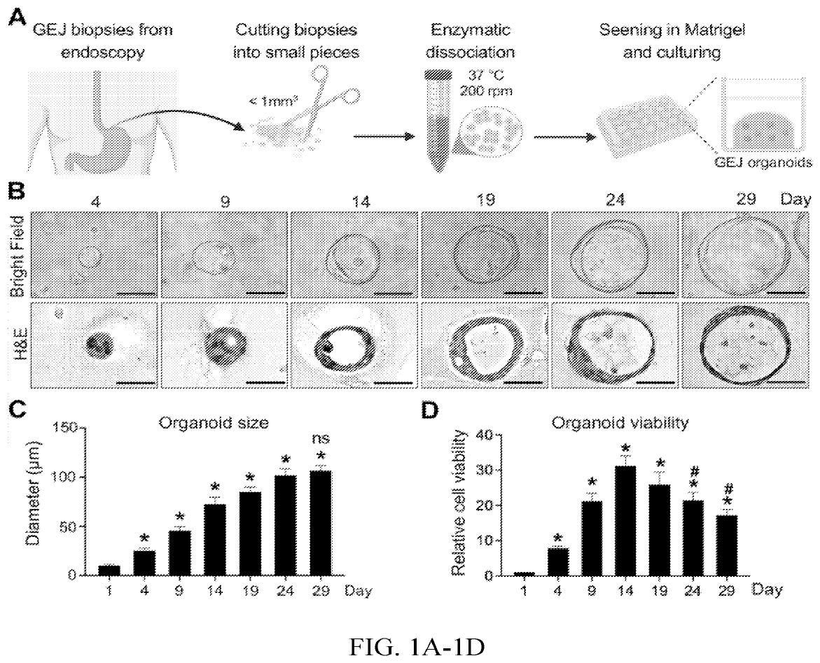

GEJ organoids were established as described in FIG. 1A. Briefly, fresh endoscopic GEJ biopsies were preserved in ice-cold conditioned PBS (PBS containing 10 μM ROCK inhibitor Y27632, 2% (vol/vol) penicillin/streptomycin, and 1× Primocin) until further processing within 24 hours. After washing biopsies with conditioned PBS at least 5 times, samples were minced into fragments <1 mm³ using microdissecting scissors. Tissue fragments were digested in DMEM containing 2.5% (vol/vol) FBS, 1% (vol/vol) penicillin/streptomycin, 1 mg/mL collagenase type IX, and 120 μg/mL dispase type II at 37° C. with 200 rpm shaking for 40-90 mins.

Following centrifugation at 400 g at 4° C. for 3 minutes, pelleted cell clusters were resuspended in Matrigel. Using 24-well plates, 2,000 cells were seeded per well in 50 μL of Matrigel. After incubating at 37° C. for 10 minutes to solidify the Matrigel, 500 μL of growth medium was added to each well. The growth medium for GEJ organoids was Advanced DMEM/F12 supplemented with 50% (vol/vol) Wnt-3A conditioned medium (home-made), 20% (vol/vol) R-spondin-1 conditioned medium (home-made), 1% (vol/vol) penicillin/streptomycin, 10 nM PGE2, 100 ng/ml human FGF-10, 50 ng/ml hEGF, 100 ng/ml Noggin, 1 mM N-acetylcysteine, 10 mM Nicotinamide, 10 nM Gastrin I, 500 nM A-83-01, 10 μM SB202190, 10 UM Y27632, 5 μM CHIR99021 (only for the first 1-2 passages), 1× Primocin, and 1× B-27 supplement. Culture medium was changed once every three days until the organoids were ready to passage.

For passaging, organoids were washed in PBS and digested with TrypLE containing 10 μM Y27632 for 5-7 mins at 37° C. After incubation, DMEM/F12 was added to stop digestion. Organoids were mechanically dissociated by pipetting and centrifuged at 500 g for 3 mins. After resuspending the pellet in Matrigel, 50-100 μL per droplet of the cell-Matrigel suspension were plated onto a new culture plate. Reagents used for organoid culture are listed in the Key Resources Table.

### Organoid Viability Assay (WST-1 Assay)

To quantify metabolically active viable cells, organoids were seeded onto 96-well plates and cultured. At indicated time points, 10 μL per well of Cell Proliferation Reagent WST-1 assay kits were added to the 96-well plates and incubated with organoids for 90 minutes. After incubation, only media was transferred to the wells of a new 96-well plate, which was read at an absorbance of 450 nm by a Thermo Scientific Microplate Reader. All experiments were performed in triplicate.

### CRISPR-Cas9 Genomic Editing of Organoids

Organoids were electroporated using the NEPA21 (Nepa Gene) system and the Alt-R CRISPR-Cas9 System (IDT). Cas9: gRNA ribonucleoprotein (RNP) complex was prepared as follows: to make the 100 μM gRNA complex, 200 μM tracrRNA labeled with ATTO™ 550 and 200 UM crRNA were mixed in equimolar concentrations, heated at 95° C. for 5 min, and then allowed to slowly cool to room temperature; to produce the RNP complex for each electroporation, 6 μL of gRNA complex (100 μM), 8.5 μg of Cas9 Nuclease (10 μg/μL), and 10.5 μL of Duplex Buffer were combined and incubated at room temperature for 10 mins. RNP complex was stored for further use at −80° C. Two days before electroporation, organoids were passaged and maintained in organoid culture medium w/o antibiotics including 5 μM CHIR99021. Organoids were dissociated into clusters of 10-15 cells, resuspended in 80 μL of Electroporation Buffer containing 4 μM Electroporation Enhancer, and then mixed with 25 μL of RNP complex targeting TP53 and 25 μL of RNP complex targeting CDKN2A. The mixture was transferred into a precooled 2-mm electroporation cuvette. Electroporation parameters were set according to Fujii et al (Fujii et al., 2015).

After electroporation, 400 μL of prewarmed culture medium including 5 μM CHIR99021 was immediately added to the electroporation cuvette. Cells were seeded after incubation for 40 min at 37° C. Two days after electroporation, transfection efficiency was measured by fluorescence microscopy. Three days after electroporation, organoids were treated with 10 μM Nutlin-3a for functional selection of TP53-mutant cells for 2-3 weeks, on the basis that Nutlin-3a inhibits the proliferation of TP53 wild-type cells. Organoids electroporated with the negative control RNP complex were used as the control group. To validate targeted mutations, genomic DNA from edited organoids was extracted, followed by PCR amplification, TOPO-cloning and Sanger sequencing. All reagents and sgRNA sequences used in this section are provided in the Key Resources Table.

### Maldi Mass Spectrometric Imaging of the Lipidome in GEJ Organoids

Organoids were transferred to even molds and immersed in M-1 Embedding Matrix after being isolated from Matrigel using Cell Recovery Solution and washed with cold PBS for 3 times. Organoid molds were wrapped in aluminum foil and floated on liquid nitrogen for progressive freezing. Frozen organoids were equilibrated to −20° C., cryosectioned at 10-μm thickness and thaw-mounted onto temperature-equilibrated, hexane-and-ethanol-washed indium tin oxide (ITO) slides (Delta Technologies, Loveland, CO) on a Leica CM1860 UV cryostat (Wetzlar, Germany). All organoids were sectioned in a layout that maximizes the number of sections per slide to compare TP53/CDKN2A^{KO} vs. control GEJ organoids. Several serial sections were cryosectioned with the same layout for technical repeats. Slides were sprayed with 40 mg/mL DHB dissolved in 70% HPLC-grade methanol/30% HPLC-grade water using an HTX-M5 sprayer (Chapel Hill, NC) with the following parameters: nozzle temperature −75° C., 8 passes, 0.1 ml/min flow rate, 1200 mm/min nozzle velocity, 3 mm track spacing, criss cross (CC) pattern, 10 psi pressure, 3 l/min gas flow rate, and 10 second drying time. The final matrix density was 8.89×10⁻³ mg/mm2 and the linear flow rate was 8.33×10-5 ml/mm.

Matrix-assisted laser desorption/ionization (MALDI) mass spectrometry imaging was acquired on Bruker MALDI TOF/TOF rapifleX instrument (Bruker Daltonik, Bremen, Germany) in the Johns Hopkins Applied Imaging Mass Spectrometry (AIMS) Core in reflectron-positive mode, at 20-micron pixel size with a 20-micron raster and 20-micron imaging laser, 200 laser shots per pixel, and a mass range of m/z 40 to 2,000. Imaging data were analyzed in SCILS Lab software (Version2020a, SCiLS GmbH, Bremen, Germany) using quantitative spectral, pixel-based, paired comparisons (Chughtai, K., et al. (2013) J. Lipid Res. 54, 333-344) between TP53/CDKN2A^{KO} and control GEJ organoids. For structural identification, m/z's of the top lipids were identified by on-tissue MS/MS using collision-induced decay (CID) with argon using the single beam laser with a resultant field of 54×54 microns with 4000 laser shots and an isolation window of +2 Da. MS/MS spectra were collected from both KO organoids. For initial identification, the Lipid Maps Structure Database (LMSD) was used by uploading a peak list, searching [M+H]⁻ and [M-Na]+ with a mass tolerance of ±0.2 m/z and all lipid classes selected. This generated a list of potential hits which were used to solve MS/MS spectra in ChemDraw Professional version 16.0.

### Genome-Wide DNA Methylation Profiling and Data Analysis

DNA methylation profiles for 4 paired sets of Control and double-knockout GEJ organoids were generated using the Illumina Methylation/EPIC array platform, which combines bisulfite conversion of genomic DNA and whole-genome amplification with direct, array-based capture and scoring of CpG loci. Genomic DNA was extracted from organoids using DNeasy Blood & Tissue kits. All DNA samples were quantified by Qubit dsDNA BR Assay, assessed for purity by A260/280 and A260/230 ratio, and examined for integrity by electrophoresis on 0.8% agarose gels. DNA samples were then hybridized to Infinium Methylation EPIC BeadChips, following the Infinium HD Methylation Assay Protocol (Moran, S., et al. (2016) Epigenomics 8, 389-399).

The SeSAME package (Zhou, W., et al., (2018). Nucleic Acids Research 46, e123) was used to extract the DNA methylation value of each probe using the openSesame function. Recommended general masking probes were removed according to the annotation file of Infinium DNA methylation arrays. Differentially methylated probes were identified by the limma package (version 3.46.0) with adjusted p-value <0.05, absolute delta methylation change >0.2.

Differentially methylated regions (DMRs) were further identified based on differentially methylated probes by the DMRcate package, with Fisher's exact test p-value<0.05.

### RNA-Sequencing (RNA-Seq) and Data Analysis

RNA sequencing was performed on 4 paired sets of Control and double-knockout GEJ organoids derived from 4 patients. Total RNA was extracted and treated with DNase I before sequencing. Libraries were constructed using NEBNet Ultra Directional RNA Library Prep kits. Quantified libraries were sequenced on the Illumina NovaSeq 6000 platform, and paired-end reads were generated. An index of the reference genome was built and pair-end clean reads were aligned to the reference genome using Hisat2 v2.0.5. Read counts for each gene were generated by FeatureCounts v1.5.0-p3. FPKM of each gene was calculated based on the length of the gene and the read count mapped to this gene. DEseq2 results were used for differential expression analysis, and genes with an adjusted P-value<0.05 and an absolute found by DESeq2 were designated as differentially expressed. Gene Ontology (GO) enrichment analysis of differentially expressed genes was implemented using the clusterProfiler R package Subramanian, A. et al. (2005) Proc. Natl. Acad. Sci. U.S.A. 102, 15545-15550.

### Real-Time Quantitative PCR

Total RNA was extracted using RNeasy kits, and DNA was eliminated via on-column DNase digestion. 500 μg RNA was reversely transcribed using iScript Select cDNA Synthesis kits (Bio-Rad). Quantitative PCR was performed using iTaq Universal SYBR Green Supermix. All results were normalized to β-actin expression. Primers used for PCR are listed in the Key Resources Table.

siRNA-Directed Gene Silencing

Two days prior to electroporation, organoids were passaged and cultured in organoid culture medium without antibiotics including 5 μM CHIR99021. Organoids were dissociated into clusters of 10-15 cells, resuspended in 100 μL of Electroporation Buffer containing 4 μM Electroporation Enhancer, and then mixed with 10 μL of 50 mM siRNA. The organoids were electroporated using the same procedure mentioned above 24 hr after electroporation. Total DNA from cloned organoids was then extracted and reversely transcribed into cDNA, followed by RT-qPCR to validate knockdown efficiency. siRNA sequences are provided in the Key Resources Table.

### Histology, Immunofluorescence and Immunohistochemistry

Organoid cultures and tissues were fixed overnight in 10% formalin at room temperature. Paraffin-embedded organoids and tissues were serially sectioned into 10-μm slices. Paraffin sections were deparaffinized, rehydrated, followed by either staining for hematoxylin and eosin (H&E), or antigen retrieval in sub-boiling 10-mM sodium citrate buffer pH 6.0 for 10 mins. For immunofluorescence (IF), slides were permeabilized in 0.5% TritonX-100 in PBS and blocked in 1% goat serum in PBS for 30 mins at room temperature. After blocking, slides were incubated with anti-Ki67 (1:200, Abcam) overnight in a humidified chamber at 4° C. Sections were washed by PBST (3 times for 5 mins each) and incubated with Alexa Fluor secondary antibodies (1:500) for 1 hr. After washing with PBST, slides were mounted with Fluoroshied with DAPI (Sigma).

Immunohistochemistry was performed using the automated Bond-Max autostainer by Leica for the following antibodies: AE1/AE3 (1:200, Santa Cruz). Images were acquired with a Lecia Inverted Confocal SP8 (Johns Hopkins Medicine Ross Imaging Center). Reagents used here are listed in the Key Resources Table.

### Chromatin Immunoprecipitation (ChIP)

ChIP experiments were performed using EZ-Magna ChIP™ A/G Chromatin Immunoprecipitation Kit (Merck Millipore, 17-10086) according to the manufacturer's procedures. Cells were crosslinked with 1% formaldehyde and lysed with cell lysis buffer containing 1× protease Inhibitor Cocktail II. Nuclei were isolated with nuclear lysis buffer supplemented with 1× protease inhibitor cocktail II. The chromatin extract was sonicated (8 mins total, AmpL 30%, pulse on 10 s, pulse off 20 s) and sheared to a length between 200 bp to 1,000 bp on wet ice. The sheared crosslinked chromatin was immunoprecipitated with antibodies co-incubated with magnetic protein A/G beads. Antibodies included anti-FOXM1 (5 μg per CHIP reaction, Invitrogen, 702664) and normal mouse IgG (1 μg per reaction). Purified DNAs were subjected to qPCR. qPCR primers are listed in the Key Resources Table.

### Xenotransplantation in Nude Mice

All procedures and experimental protocols involving mice were approved by the Animal Experimental Committee of the Johns Hopkins University School of Medicine. For xenotransplantation of organoids, 2×10⁶ organoid cells or ESO26 cells resuspended in cold 50% Matrigel were injected into the axillary of nude mice. For the WEB2086 treatment assay, 5 mg/kg.d of WEB2086 or vehicle control (1.25% DMSO in PBS) was administered by intraperitoneal injection every two days for 3 weeks. Xenograft size was measured twice a week. The xenograft volume (V) was monitored by measuring the length (L) and width (W) with a caliper based on the formula V=1/2×(L×W²). Mice were sacrificed at the end of the experiment, and xenografts were excised for further analyses.

### Statistical Analysis

The data are presented as mean±SD unless indicated otherwise. Statistical analysis was assessed using GraphPad Prism 9.2. For in vitro experiments, student's t-test or one-way ANOVA was used unless otherwise noted in the figure legends. For xenograft experiments, unpaired Student's t-test. P<0.05 was considered statistically significant.

### Results

### Establishment and Characterization of Human Normal GEJ Organoids:

To address the paucity of biologically relevant GEJ-specific disease models, GEJ 3D organoids were generated from human primary endoscopic GEJ biopsies, which were confirmed pathologically to contain neither dysplastic nor neoplastic cells. Freshly isolated GEJ crypts were embedded in Matrigel and incubated with conditioned medium containing stem-critical growth factors (FIG. 1A). Under established culture conditions, 3D organoids were generated from 6 normal GEJ biopsies, with a 100% success rate. These organoids were characterized using phase-contrast imaging, hematoxylin/eosin (H&E) staining, and viability (WST-1) assays (FIG. 1B-D). At day 4 after initial seeding, 3D spherical structures were formed and reached 25 μm in diameter. These structures continued to grow, eventually reaching a size plateau at 106 μm between days 24 and 29 (FIGS. 1B and C). These GEJ organoids consisted of 120-250 cells at day 24, indicating that population doubling time in culture was 77.85±5.54 hrs. WST-1 assays showed that cell viability in organoids increased to a peak on day 14 and significantly diminished by day 24 (FIG. 1D). Organoids derived from 6 independent normal GEJ biopsies were monitored; they propagated continuously for 4 to 6 months ex vivo.

### TP53/CDKN2A Loss Promotes Proliferation, Dysplasia, and Neoplastic Transformation of GEJ Organoids:

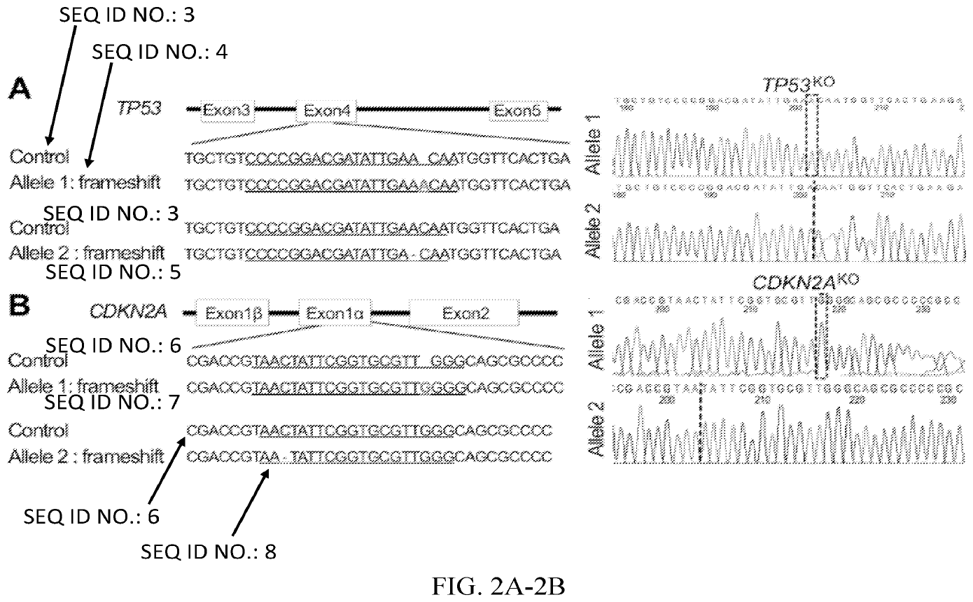

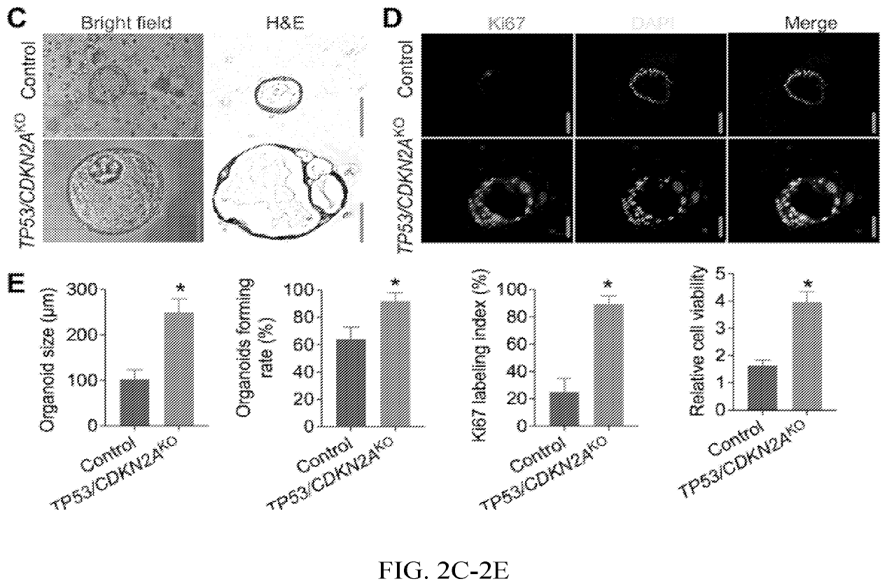

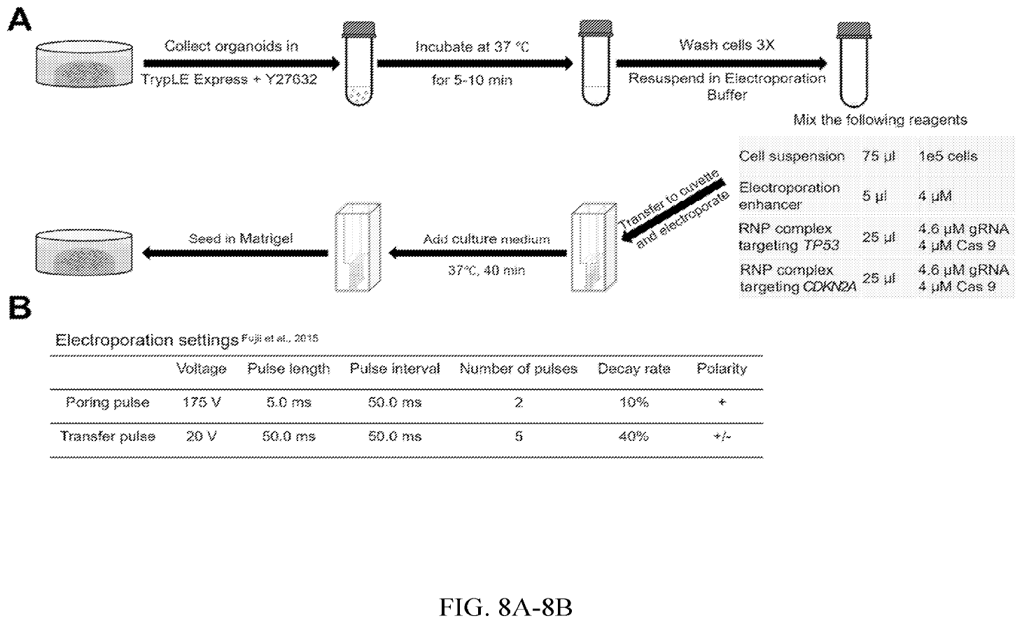

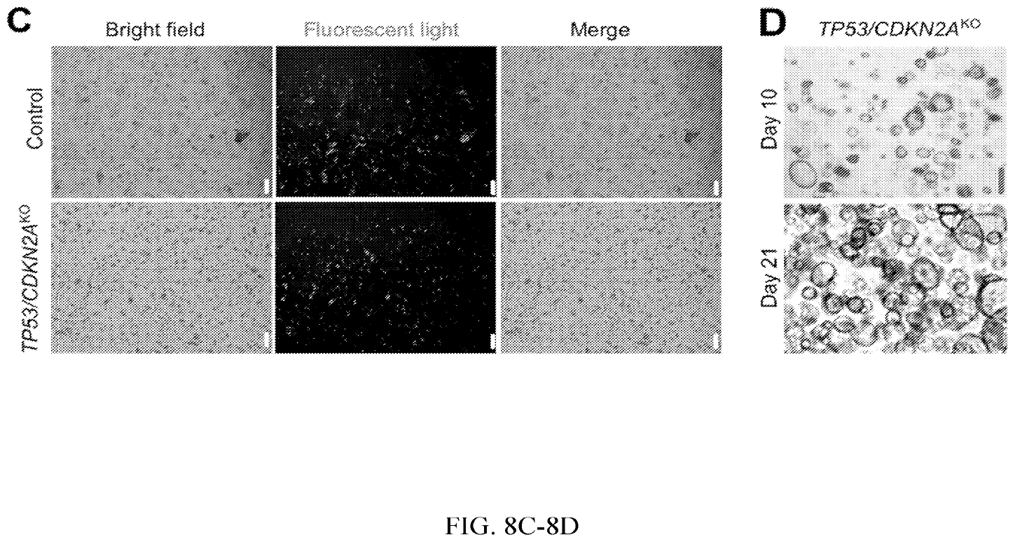

Next, a model that could grow more vigorously, survive longer in culture, and better facilitate studies of GEJ neoplastic transformation was generated. TP53 and CDKN2A were inactivated in the GEJ organoid model using the CRISPR-Cas9 genome editing system (IDT Alt-R). An all-in-one Cas9: gRNA ribonucleoprotein (RNP) complex targeting TP53 (exon 4) and CDKN2A (exon 1α) was prepared using a gRNA complex (which combines crRNA and tracrRNA) and Cas9 nuclease. Human GEJ organoids were then dissociated into small cell clusters (5-15 cells each) and delivered the RNP complex by electroporation using an optimized protocol (Methods) (FIG. 8A). For the control organoid group, a negative control RNP complex was electroporated. The transfection showed high electroporation efficiency of 42% (FIG. 8B). Subsequently, organoids with mutant-TP53 were selected for using Nutlin-3a, which inhibits MDM2 and hence induces growth arrest of TP53-wildtype cells (FIG. 8C). Sanger sequencing was performed to validate specific editing of targeted TP53 and CDKN2A exons. Frameshift mutations, including 1-bp insertions or deletions at the TP53 and CDKN2A target sites (FIG. 2A and FIG. 2B), were observed, verifying successful genome-editing in GEJ organoids.

Phenotypic changes of GEJ organoids upon loss of TP53 and CDKN2A was characterized. Control organoids formed a single layer of epithelial cells with normal nuclei at day 10 after seeding. In sharp contrast, TP53/CDKN2A^{KO} organoids exhibited substantially larger diameters, more complex multicellular structures, increased mitotic, and markedly enlarged, atypical nuclei consistent with dysplastic morphology (FIG. 2C and FIG. 2D). Organoid-forming rate also increased significantly in TP53/CDKN2A^{KO} relative to control organoids (92% vs. 64%, FIG. 2E). Immunofluorescence (IF) staining showed a striking elevation of Ki67 labeling index in TP53/CDKN2A^{KO} vs. control GEJ organoids (89.4% vs. 24.9%, FIG. 2D and FIG. 2E). Consistently, WST-1 assays revealed a 2.4-fold increase in proliferation of TP53/CDKN2A^{KO} vs. control organoids (FIG. 2E). Furthermore, control organoids could only be continuously propagated for up to 6 months, while TP53/CDKN2A^{KO} organoids were propagated for more than 18 months, with both cultures split biweekly at 1:2. These data demonstrate that loss of TP53/CDKN2A potently enhances proliferation and dysplasia of GEJ organoids in vitro. To assess the in vivo effect of TP53/CDKN2A inactivation in GEJ organoids, xenotransplantation assays were performed. Control and TP53/CDKN2A^{KO} organoid cells (2×10⁶ cells/injection) were subcutaneously injected into the left and right armpit of 5 nude mice respectively. Within a 5-month post-injection observation interval, no tumors were formed in 5 mice injected with control GEJ organoids. In contrast, TP53/CDKN2A^{KO} organoids developed tumors in 3 of 5 injected mice within 8 weeks (FIG. 2F). These tumors appeared morphologically similar to highly differentiated gastroesophageal adenocarcinoma. H&E and IHC analysis showed enlarged nucleus, increased mitosis, and positive expression of AE1/AE3 in xenografts arising 8 from TP53/CDKN2A^{KO} organoids (FIG. 2G). Thus, these data in vivo suggest that inactivation of TP53/CDKN2A promotes neoplastic properties in GEJ organoids.

### Lipidomic MALDI Imaging MS (IMS) Identifies PTAFs as Top Upregulated Lipids in TP53/CDKN2A^{KO} Organoids:

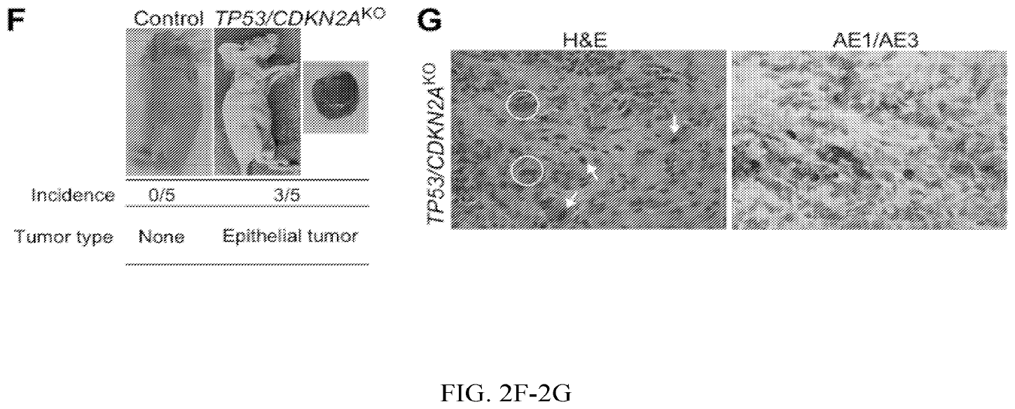

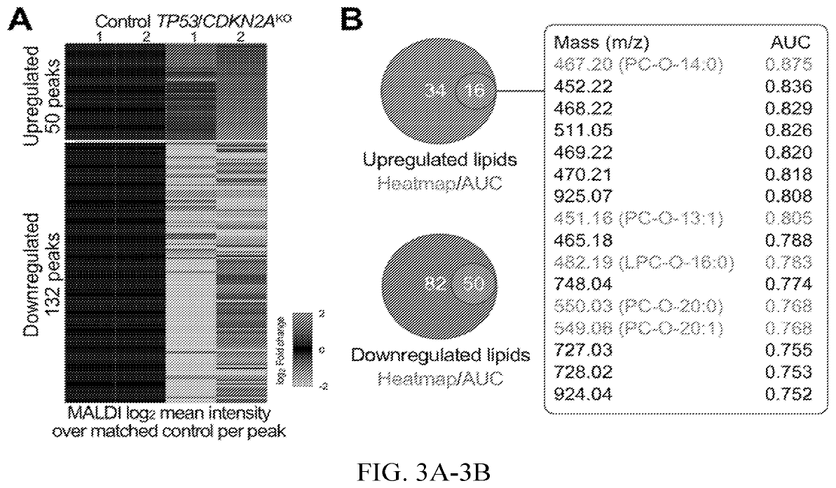

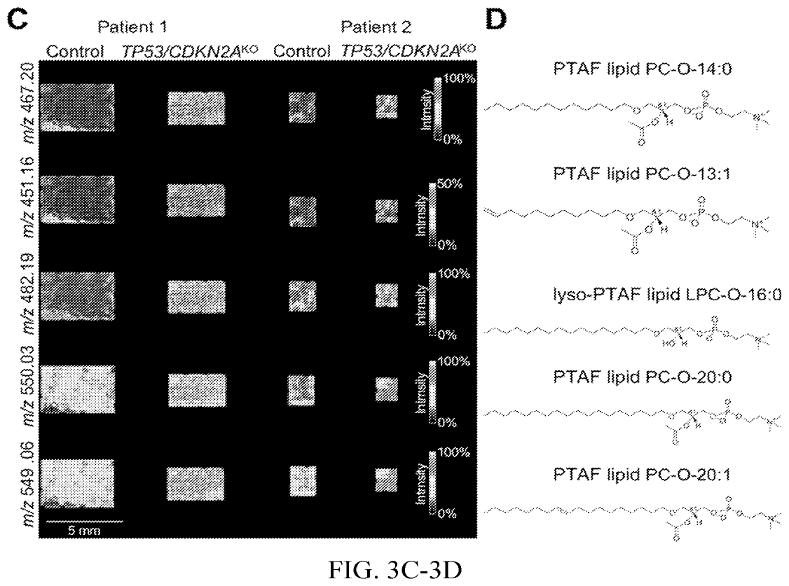

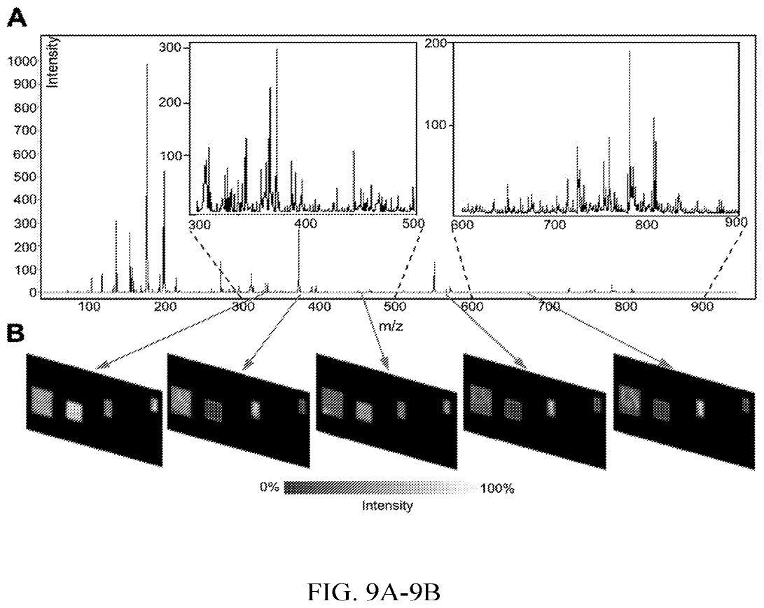

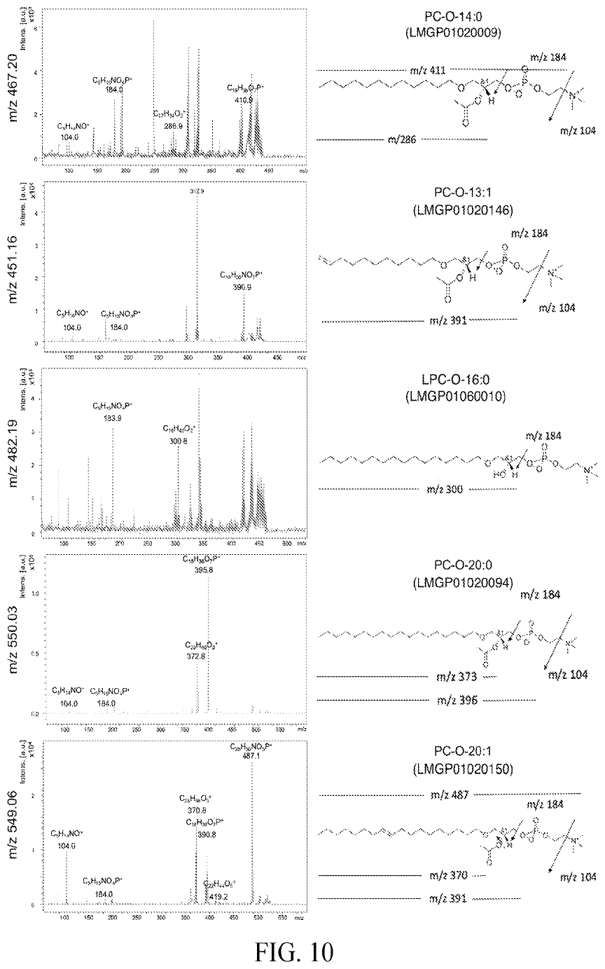

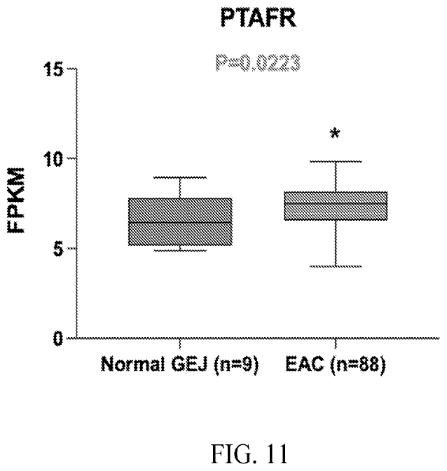

Reprogramming and dysregulation of lipid metabolism is a hallmark of cancer (Li et al.). However, it is unknown if and how lipid metabolic processes are altered during early GEJ carcinogenesis. To address this knowledge gap, lipidomic MALDI-IMS was applied to discover altered lipid species in TP53/CDKN2A^{KO} vs. control organoids. Within the mass range from m/z 40 to 2,000, mass spectra of lipid species was obtained through direct analysis of organoid sections, while ion images were generated from each peak and displayed as the position in the organoid section and relative intensity (FIG. 9A and FIG. 9B). A cutoff of m/z >450 was applied to minimize the background matrix signal in imaging experiments, as well as focus on phospholipids which are generally larger than m/z 400. Analysis based on fold-changes of mean intensity data for individual peaks identified 50 upregulated peaks and 132 downregulated peaks in TP53/CDKN2A^{KO} vs. control organoids under average fold-change >1.5 (FIG. 3A). In parallel, receiver operating characteristic (ROC) analysis was performed, using an area-under-the-curve (AUC) threshold value of >0.75. By this criterion, 16 and 50 peaks were respectively upregulated and downregulated in TP53/CDKN2A^{KO} vs. control organoids. (FIG. 3B). Concordantly, all of these altered peaks identified by the ROC method were dysregulated in the same directions based on their fold-changes (FIG. 3B). On-organoid MS/MS (FIG. 10) identified that among the 16 shared upregulated peaks, the one with the highest AUC (m/z 467.20) was a specific platelet-activating factor (PTAF) lipid PC-O-14:0 (LMGP01020009). Moreover, 4 additional lipids were identified as either PTAF lipids (m/z 451.16, PC-O-13:1, LMGP01020146; m/z 550.03, PC-O-20:0, LMGP01020094; and m/z 549.06, PC-O-20:1, LMGP01020146) or a precursor of a PTAF lipid (m/z 482.19, LPC-O-16:0, LMGP01060010). MALDI imaging data and chemical structures of these lipids are displayed in FIG. 3C and FIG. 3D. Interestingly, PTAF lipids are a family of glycerophosphocholines implicated as bioactive mediators in diverse pathologic processes, including tumor angiogenesis and metastasis (Melnikova and Bar-Eli, 2007; Tsoupras et al., 2009).

### Inhibition of PTAF/PTAFR Suppresses Growth and Proliferation of TP53/CDKN2A^{KO} GEJ Organoids:

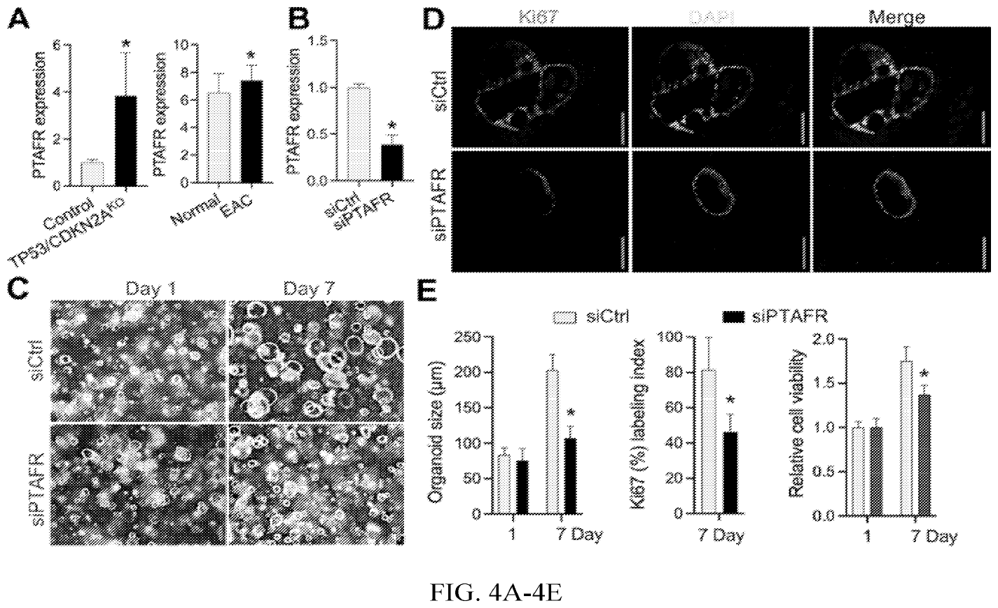

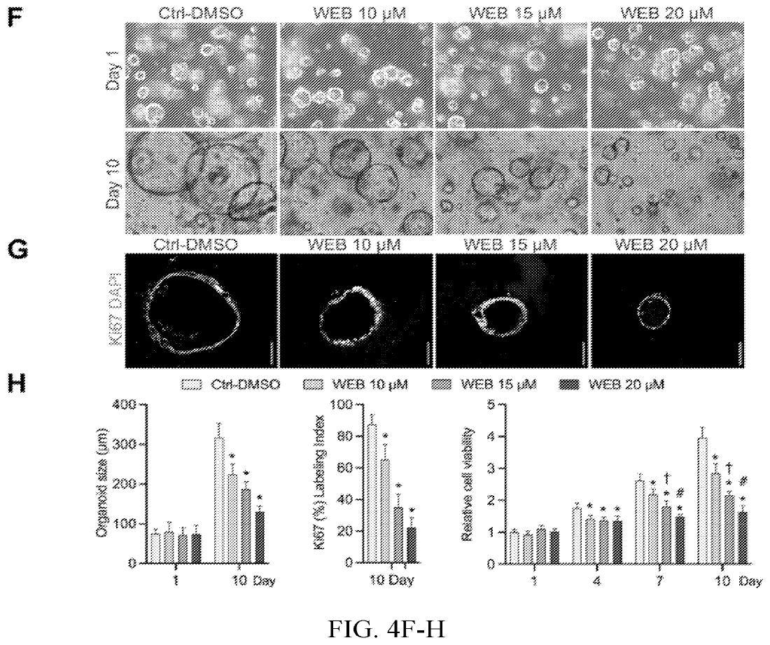

Following identification of multiple PTAF as the notably increased phospholipids in TP53/CDKN2A^{KO} GEJ organoids, its potential mechanisms in GEJ neoplasia development were addressed. As a glycerophosphocholine, PTAF exerts biological effects by binding to its cognate receptor, PTAFR (Ishii et al., 2002). Thus, PTAFR levels in GEJ neoplasia, both in the organoid model and in The Cancer Genome Atlas (TCGA), were first evaluated. Like its cognate lipid ligand, PTAFR expression was upregulated in TP53/CDKN2A^{KO} vs. control organoids (FIG. 4A). Consistent with this finding, TCGA EAC tumors exhibited significantly higher PTAFR mRNA levels than did nonmalignant TCGA GEJ samples (FIG. 4A). PTAF/PTAFR function in the early GEJ model system was directly abrogated by siRNA knockdown and pharmacologic inhibition. Importantly, silencing PTAFR expression significantly decreased average size, cell viability, and Ki-67 index of TP53/CDKN2A^{KO} relative to control organoids (FIGS. 4B-E). In parallel, TP53/CDKN2A^{KO} organoids were treated with either vehicle control (0.1% DMSO) or a specific PTAFR pharmacologic antagonist, WEB2086, at various concentrations. In agreement with the siRNA results, TP53/CDKN2A^{KO} organoids displayed significantly reduced size and Ki-67 labeling index after WEB2086 treatment (FIG. 4F to FIG. 4H). WST-1 assays showed that metabolically active cells began to decrease on day 4, and a time- and dose-dependent inhibitory effect was confirmed on day 7 and 10 (FIG. 4H).

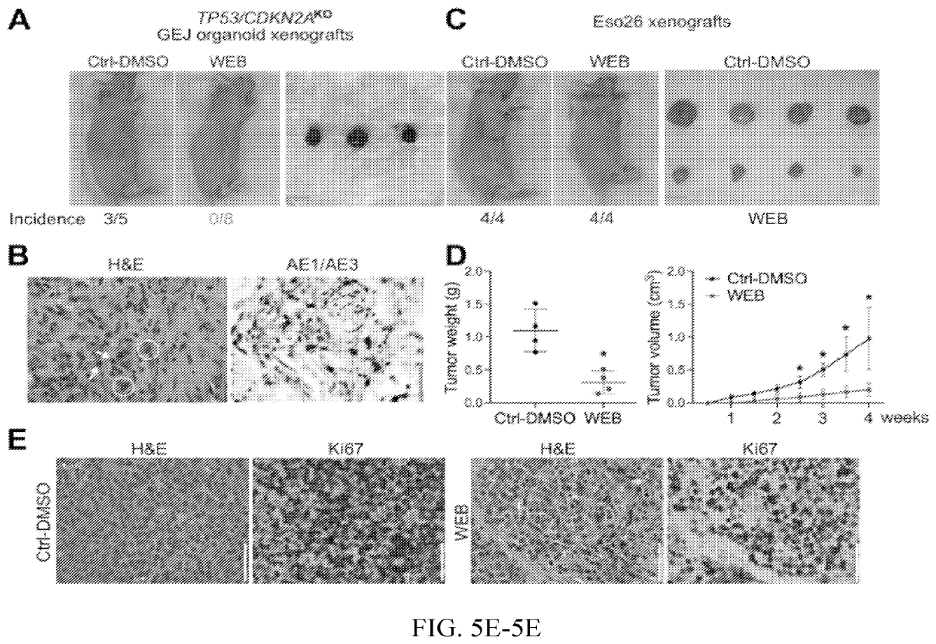

Next, it was explored whether these effects could be replicated in vivo. TP53/CDKN2A^{KO} organoid cells (2×10⁶ cells/injection) were subcutaneously injected into the armpit of nude mice. Mice were treated with vehicle control (1.25% DMSO) or WEB2086. Within a 3-month post-injection observation interval, 3 out of 5 injected mice developed tumors in the Ctrl-DMSO group within 7 weeks (FIG. 5A). Importantly, WEB2086 treatment completely prevented tumor formation in TP53/CDKN2A^{KO} organoids (FIG. 5A). The effect of WEB2086 was also assessed in a xenograft model derived from an established EAC cell line (Eso26); in this model as well, PTAFR inhibition resulted in significant suppression of EAC xenograft growth and weight (FIG. 5C and FIG. 5D). IHC analysis showed downregulated expression of Ki67 in WEB2086 treated Eso26 tumor xenografts (FIG. 5E). Taken together, these results establish that blockade of the PTAF/PTAFR lipid metabolic cascade potently inhibits GEJ organoid growth, proliferation, and tumorigenesis in vitro and in vivo, suggesting an important function for PTAF/PTAFR in mediating neoplastic progression at the GEJ.

### Inactivation of TP53/CDKN2A Alters the Methylome and Transcriptome of GEJ Organoids, Partially Mediated by FOXM1:

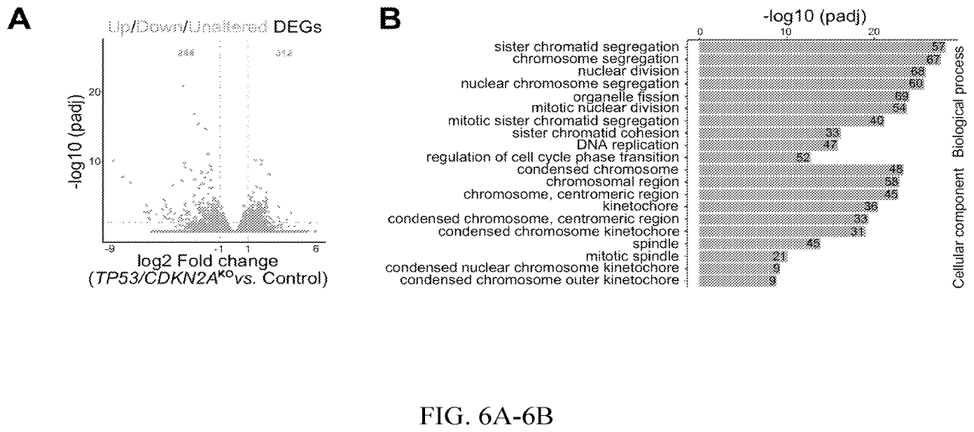

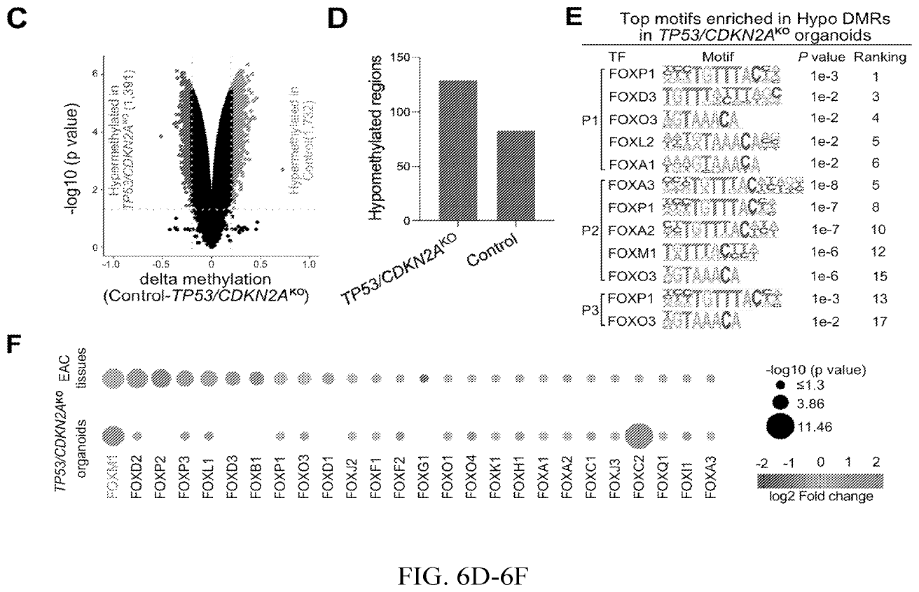

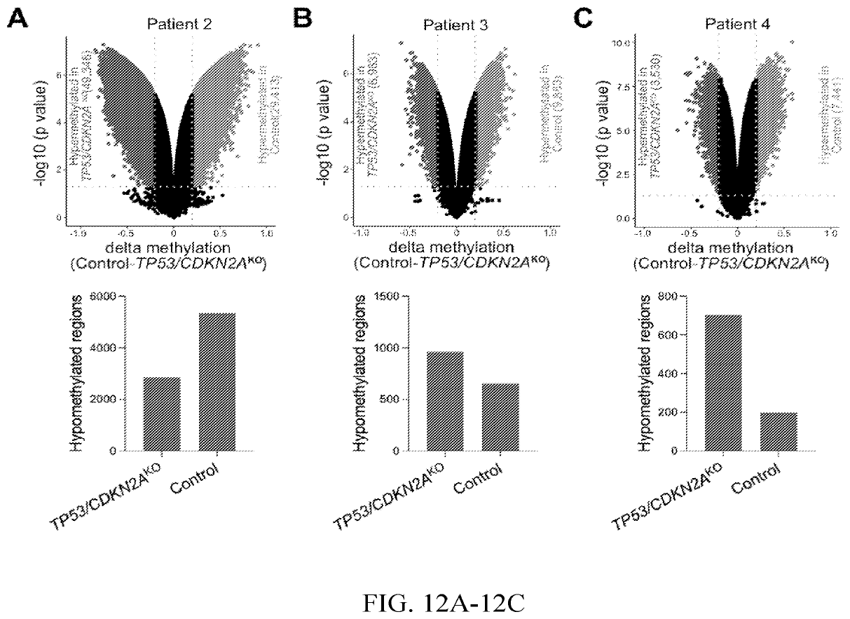

To elucidate the comprehensive epigenomic and transcriptomic differences between control and TP53/CDKN2A^{KO} organoids, transcriptome sequencing (RNA-seq) and Illumina Methylation EPIC array profiling were applied respectively. Specifically, paired wild-type and TP53/CDKN2A^{KO} organoids from 4 patients were subjected to RNA-seq. Compared with the control group, TP53/CDKN2A^{KO} organoids contained 556 significantly differentially expressed genes (312 upregulated and 244 downregulated; FIG. 6A). Gene Ontology analysis of these genes identified strong enrichment of biological processes and pathways related to mitotic entry and cell cycle progression (FIG. 6B). At the epigenomic level, differentially methylated regions (DMRs) between control and TP53/CDKN2A^{KO} organoids derived from 4 patients were determined using the Dmrseq package (Korthauer, K., (2019) Biostatistics 20, 367-383) with a cutoff p-value of <0.05 and an absolute delta-methylation value of >0.2. In organoids derived from patient 1, a total of 1,732 CpG sites were significantly hypermethylated in the control group, while 1,391 CpG sites were significantly hypermethylated in TP53/CDKN2A^{KO} organoids (FIG. 6C); Furthermore, 129 and 83 hypomethylated regions were confirmed in TP53/CDKN2A^{KO} and control organoids, respectively (FIG. 6D). Results from organoids derived from the other three patients were shown in FIG. 12. DNA hypomethylated regions contain regulatory elements associated with the binding of transcription factors (TFs) (Héberlé and Bardet, 2019). To identify candidate TF regulators in the neoplastic GEJ organoid model, enriched TF-recognition motif sequences in hypomethylated DMRs in TP53/CDKN2A^{KO} organoids were investigated using the HOMER package (Heinz et al., 2010). Notably, motifs of the Forkhead box (FOX) TF family were among the most enriched sequences in hypomethylated DMRs in TP53/CDKN2A^{KO} organoids (FIG. 6E). Because different FOX TF family members recognize similar motif sequences (Obsil and Obsilova, 2008), screening analysis of the expression of 26 FOX TFs based on RNA-seq data from organoids was performed, as well as on The Cancer Genome Atlas (TCGA) human GEJ and EAC sample data. Both FOXM1 and FOXC2 were strongly increased in TP53/CDKN2A^{KO} organoids. FOXM1 was also upregulated in EAC vs. normal GEJ tissues (FIG. 6F), suggesting it as a candidate TF enriched in hypomethylated DMRs with possibly increased activity in TP53/CDKN2A^{KO} organoids. FOXM1 is a regulator of cell proliferation and cell cycle progression in cancer, in line with the pro-neoplastic phenotypes in TP53/CDKN2A^{KO} organoids.

### PTAFR is a Direct Downstream Target of FOXM1:

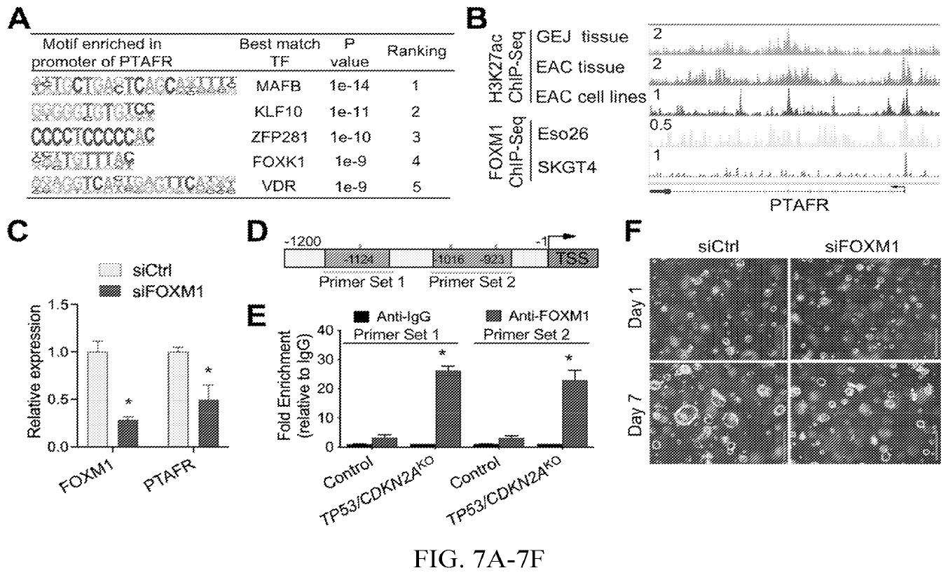

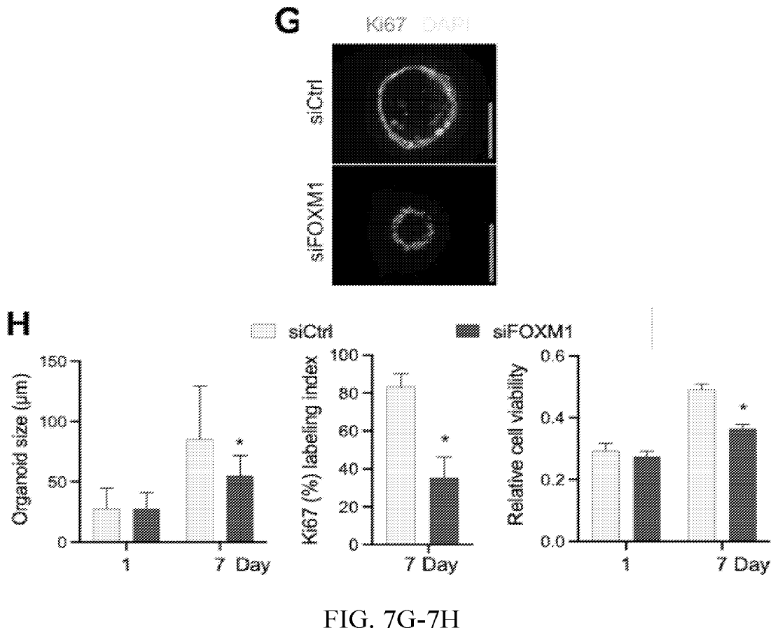

PTAFs phospholipids were identified as one of the most induced classes of lipid molecules in lipidomic profiling, and the upregulation of its cognate receptor, PTAFR, in TP53/CDKN2A^{KO} GEJ organoids (FIG. 4). To further elucidate mechanisms mediating increased PTAFR expression in TP53/CDKN2A^{KO} organoids, motif enrichment analysis of the PTAFR promoter, which revealed that the FOX motif sequence again was highly enriched (No. 4) (FIG. 7A). This was particularly interesting given above data identifying FOXM1 as a potential regulator involved in epigenetic alterations in TP53/CDKN2A^{KO} organoids.

To validate whether FOXM1 regulates the transcription of PTAFR, CHIP-seq was performed to comprehensively screen for transcription factor activation of PTAFR promoter regions in both primary EAC tissues and EAC-derived cell lines. Indeed, FOXM1 occupied the promoter of PTAFR in two different EAC cell lines (FIG. 7B). Moreover, H3k27ac ChIP-seq data (30) showed that PTAFR harbors strong H3k27ac signals at its promoter and candidate enhancers in both EAC primary tumors and cell lines, suggesting robust transcriptional activation. In contrast, in normal GEJ samples, H3k27ac was barely deposited at the PTAFR locus, indicating weak/inactive transcription (FIG. 7B).

Since the above ChIP-seq data were generated in EAC cell lines and primary tumors (FIG. 7A), FOXM1 ChIP-qPCR in GEJ organoids was performed. Importantly, endogenous FOXM1 showed prominent occupancy at the PTAFR promoter in TP53/CDKN2A^{KO}, with a more than 7.9-fold increase in enrichment compared to wild-type GEJ organoids (FIG. 7D and FIG. 7E). To validate the regulation of FOXM1 upon PTAFR transcription, FOXM1 was silenced via siRNA in TP53/CDKN2A^{KO} organoids, which showed that FOXM1 knockdown significantly reduced PTAFR expression (FIG. 7C). In addition, FOXM1 silencing reduced average organoid size, cell viability, and Ki67 index in TP53/CDKN2A^{KO} organoids (FIG. 7F to FIG. 7H), phenocopying the effects obtained with PTAF/PTAFR blockade. These data demonstrate that FOXM1 binds directly to the PTAFR promoter, thereby augmenting PTAFR expression and proliferation in TP53/CDKN2A^{KO} organoids.

### Discussion

A major hurdle in understanding the molecular origins and biology of GEJ cancer is a paucity of appropriate biologically relevant models. Described herein is the development and use of a human normal GEJ-derived organoid culture model. This model is a desirable system for studying critical properties of the original native tissue in vitro, including morphological, histological, and molecular features. This model was developed using a highly reliable protocol ensuring successful organoid culture from endoscopic biopsies. Wild-type GEJ organoids described herein can be propagated in vitro for at least 4 months, and CRISPR-edited organoids even longer (for at least 18 months). This platform offers great promise in modeling GEJ-associated diseases, characterizing normal and diseased GEJ conditions, and discovering novel molecular mechanisms underlying the transition from normal to diseased GEJ.

CRISPR-engineered human TP53/CDKN2A^{KO} organoids described herein offer a productive tool for modeling early neoplastic events at the GEJ. The dynamic model described herein newly demonstrates that TP53/CDKN2A inactivation directly causes biologic and molecular features consistent with GEJ neoplastic progression. Moreover, TP53/CDKN2A inactivation directly causes abnormal lipidomic changes in GEJ organoids. The lipids most upregulated by TP53/CDKN2A^{KO} in GEJ organoids include several PTAFs, a family of phospholipid mediators. PTAF/PTAFR is shown herein to be an etiologic mediator in neoplastic progression induced by TP53/CDKN2A^{KO} at the GEJ. The results presented herein demonstrate the therapeutic potential of PTAF/PTAFR inhibition during GEJ neoplastic progression.

As shown herein, the PTAFR antagonist WEB2086 inhibited early neoplastic changes in TP53/CDKN2A^{KO} organoids, while causing analogous growth suppression in ESO26 esophageal adenocarcinoma cells. TP53/CDKN2A^{KO} elicits extensive epigenetic and transcriptional programs that propel the normal GEJ toward a malignant state. Here, integrative epigenetic and transcriptional analyses now demonstrate enrichment in oncogenic transcription factors, including FOXM1, directly caused by TP53/CDKN2A inactivation.

FOXM1 is overexpressed in multiple solid tumors and signaling downstream of this transcription factor contributes to cancer development and progression via cross-talk with multiple cell signaling pathways, particularly PI3K/Akt, NF-κB, EGFR, MAPK, and sonic hedgehog. novel mechanistic link between FOXM1 and PTAFR is shown herein: FOXM1 binds to the PTAFR gene promoter, thereby upregulating PTAFR expression. Consistent with results after PTAF/PTAFR inhibition, FOXM1 downregulation in TP53/CDKN2A^{KO} GEJ organoids leads to severe cell growth inhibition.

In summary, the human primary benign GEJ organoid model described herein and its pro-neoplastic induction by TP53/CDKN2A knockout now enables deconstruction of early GEJ tumorigenesis and neoplastic progression. Furthermore, lipidomic, epigenetic, and transcriptional profiling studies yield valuable insights into mechanistic underpinnings of GEJ malignancy. The highly induced phospholipid family, PTAFs, and their receptor, PTAFR, are upregulated by FOXM1 and show strong pro-neoplastic activity in GEJ evolution while simultaneously revealing potential targeted therapeutic strategies against GEJ cancers. Organoids derived from human primary normal may be used to model other cancer-associated genomic loci, as well as other types of malignancy, where they may expand understanding of specific gene-regulatory networks and yield novel potential therapeutic targets.

### References

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Although the foregoing subject matter has been described in some detail by way of illustration and example for purposes of clarity of understanding, it will be understood by those skilled in the art that certain changes and modifications can be practiced within the scope of the appended claims.

1. A gastroesophageal junction (GEJ) organoid, the organoid comprising cells wherein expression of tumor protein P53 (TP53) and cyclin dependent kinase inhibitor 2A (CDKN2A) is reduced compared to expression in a wildtype GEJ cell.

2. The GEJ organoid of claim 1, wherein the organoid is capable of in vitro propagation for at least 12 months.

3. The GEJ organoid of claim 1, wherein the organoid is capable of in vitro propagation for at least 18 months.

4. The GEJ organoid of any one of claims 1-3, wherein the organoid is generated by a method comprising culturing cells derived from the gastroesophageal junction of a subject in a medium comprising prostaglandin E 2 (PGE2), human fibroblast growth factor-10 (rFGF-10), human epidermal growth factor (hEGF), Noggin, and Gastrin I.

5. The GEJ organoid of claim 4, wherein the medium further comprises one or more inhibitors, wherein the one or more inhibitors are selected from ALK5 inhibitor A-83-01, p38 MAPK inhibitor SB202190, Rho-associated, coiled-coil containing protein kinase (ROCK) inhibitor Y27632, and GSK3 inhibitor CHIR99021.

6. The GEJ organoid of claim 4 or claim 5, wherein the medium further comprises one or more supplements selected from antibiotics, N-acetylcysteine, and Nicotinamide.

7. The GEJ organoid of any one of the preceding claims, wherein expression of TP53 and CDKN2A is reduced by CRISPR-mediated gene inactivation or siRNA mediated gene silencing.

8. A method of generating a gastroesophageal junction organoid, the method comprising:
a) culturing cells obtained from the gastroesophageal junction of a subject in a suitable medium to promote cell growth and/or differentiation; and
b) reducing expression of tumor protein P53 (TP53) and cyclin dependent kinase inhibitor 2A (CDKN2A) in the cells.

9. The method of claim 8, wherein the subject is a human.

10. The method of claim 8 or claim 9, wherein expression of TP53 and CDKN2A is reduced by CRISPR-mediated gene inactivation.

11. The method of claim 10, wherein expression of TP53 and CDKN2A is reduced by transfecting cells with a TP53 crRNA comprising the sequence CCCCGGACGATATTGAACAA (SEQ ID NO: 1), a CDKN2A crRNA comprising the sequence CCCAACGCACCGAATAGTTA (SEQ ID NO: 2), and a nuclease.

12. The method of claim 11, wherein the nuclease comprises Cas9 endonuclease.

13. The method of claim 8 or claim 9, wherein expression of TP53 and CDKN2A is reduced by si-RNA mediated gene silencing.

14. The method of any one of claims 8-13, wherein the medium comprises prostaglandin E 2 (PGE2), human fibroblast growth factor-10 (rFGF-10), human epidermal growth factor (hEGF), Noggin, and Gastrin I.

15. The method of claim 14, wherein the medium further comprises one or more inhibitors, wherein the one or more inhibitors are selected from ALK5 inhibitor A-83-01, p38 MAPK inhibitor SB202190, Rho-associated, coiled-coil containing protein kinase (ROCK) inhibitor Y27632, and GSK3 inhibitor CHIR99021.

16. The method of claim 14 or claim 15, wherein the medium further comprises one or more supplements selected from antibiotics, N-acetylcysteine, and Nicotinamide.

17. The GEJ organoid of any one of the claims 1-7, for use in an in vitro method of evaluating a potential anti-cancer agent.

18. An in vitro method of evaluating a potential anti-cancer agent, the method comprising:
a) contacting the GEJ organoid of any one of claims 1-7 with the potential anti-cancer agent; and
b) measuring a response in the organoid.

19. The method of claim 18, wherein measuring a response in the organoid comprises measuring organoid size, cell viability, and/or one or more markers of cell proliferation.

20. The method of claim 19, wherein a decreased organoid size, a decreased cell viability, and/or decreased expression of one or more markers of cell proliferation following contacting the organoid with the potential anti-cancer agent indicates a positive response to the agent.

21. A method of treating cancer in a subject, comprising providing to the subject a Platelet Activating Factor Receptor (PTAFR) antagonist.

22. The method of claim 21, wherein the PTAFR antagonist comprises 4-[3-[4 [(2-Chlorophenyl)-9-methyl-6H-thieno[3,2-f][1,2,4]triazolo[4,3-α]diazepin-2-yl]-1-oxopropyl]morpholine (WEB2086).

23. The method of claim 21 or claim 22, wherein the cancer is gastroesophageal junction adenocarcinoma.

24. Use of the GEJ organoid of any one of claims 1-7 in a method of evaluating a potential anti-cancer agent.

25. Use of claim 24, wherein the method comprises:
a) contacting the GEJ organoid of any one of claims 1-7 with the potential anti-cancer agent; and
b) measuring a response in the organoid.

26. The use of claim 25, wherein measuring a response in the organoid comprises measuring organoid size, cell viability, and/or one or more markers of cell proliferation.

27. The use of claim 26, wherein a decreased organoid size, a decreased cell viability, and/or decreased expression of one or more markers of cell proliferation following contacting the organoid with the potential anti-cancer agent indicates a positive response to the agent.

28. Use of a platelet activating factor receptor (PTAFR) antagonist in a method of treating cancer in a subject.

29. The use of claim 28, wherein the PTAFR antagonist comprises 4-[3-[4 [(2-Chlorophenyl)-9-methyl-6H-thieno[3,2-f][1,2,4]triazolo[4,3-α]diazepin-2-yl]-1-oxopropyl]morpholine (WEB2086).

30. The use of claim 28 or claim 29, wherein the cancer is gastroesophageal junction adenocarcinoma.


SEQUENCE LISTING (8 sequences)

SEQ ID NO: 1 - DNA, 20 bp, Synthetic construct
  source 1..20: mol_type = other DNA
ccccggacgatattgaacaa

SEQ ID NO: 2 - DNA, 20 bp, Synthetic construct
  source 1..20: mol_type = other DNA
cccaacgcaccgaatagtta

SEQ ID NO: 3 - DNA, 37 bp, synthetic construct
  source 1..37: mol_type = other DNA
tgctgtccccggacgatattgaacaatggttcactga

SEQ ID NO: 4 - DNA, 38 bp, synthetic construct
  source 1..38: mol_type = other DNA
tgctgtccccggacgatattgaaacaatggttcactga

SEQ ID NO: 5 - DNA, 36 bp, synthetic construct
  source 1..36: mol_type = other DNA
tgctgtccccggacgatattgacaatggttcactga

SEQ ID NO: 6 - DNA, 35 bp, synthetic construct
  source 1..35: mol_type = other DNA
cgaccgtaactattcggtgcgttgggcagcgcccc

SEQ ID NO: 7 - DNA, 36 bp, synthetic construct
  source 1..36: mol_type = other DNA
cgaccgtaactattcggtgcgttggggcagcgcccc

SEQ ID NO: 8 - DNA, 34 bp, synthetic construct
  source 1..34: mol_type = other DNA
cgaccgtaatattcggtgcgttgggcagcgcccc